(19) United States
(12) Patent Application Publication
(10) Pub. No.: US 2024/0285760 A1
(43) Pub. Date: Aug. 29, 2024

(54) METHODS AND COMPOSITIONS FOR REMOTE CONTROL OF T CELL THERAPIES BY THERMAL TARGETING

(71) Applicant: GEORGIA TECH RESEARCH CORPORATION, Atlanta, GA (US)

(72) Inventors: Gabriel A. KWONG, Atlanta, GA (US); Marielena GAMBOA, Atlanta, GA (US); Ian C. MILLER, Atlanta, GA (US); Ali ZAMAT, Atlanta, GA (US)

(21) Appl. No.: 18/573,858

(22) PCT Filed: Jun. 24, 2022
(86) PCT No.: PCT/US2022/034958

§ 371 Date: Dec. 22, 2023

Related U.S. Application Data
(60) Provisional application No. 63/214,761, filed on Jun. 24, 2021.

(51) Int. Cl. A61K 39/00; A61P 35/00; C07K 14/54; C12N 5/0783; C12N 15/85

(52) U.S. Cl. CPC A61K 39/4635; A61K 39/4611; A61K 39/4633; A61K 39/464412; A61P 35/00; C07K 14/5443; C12N 5/0636; C12N 15/85; A61K 2239/39; C12N 2510/00; C12N 2800/107; C12N 2830/002

(57) ABSTRACT

The present disclosure relates to promoter constructs comprising: one or more heat shock elements: a core promoter; and a gene of interest: vectors comprising the promoter constructs, and immune cells modified to include the promoter constructs. The promoter constructs provide the ability to remotely control immune cell therapies by thermal targeting. The present disclosure also provides methods of use for the promoter constructs.

This application claims the benefit of U.S. Provisional Application No. 63/214,761, filed on Jun. 24, 2021, which is incorporated herein by reference in its entirety.

This invention was made with government support under grant number DP2HD091793 awarded by the National Institutes of Health. The government has certain rights in the invention.

### BACKGROUND

Engineered T cell therapies such as Chimeric Antigen Receptor (CAR) T cells are transforming clinical care for hematological malignancies, spurring numerous efforts to expand their use for different cancer types and applications. However, this success has not reliably translated to solid tumors. The factors that contribute to low response rates are multifaceted and include the paucity of tumor-specific antigens, inefficient persistence and expansion of adoptively transferred T cells, and immunosuppression by the tumor microenvironment (TME). Promising approaches to improve anti-tumor activity of engineered T cells include systemic administration of potent immunostimulatory agents such as cytokines, checkpoint blockade inhibitor antibodies, and bispecific T cell engagers (BiTEs). However, these biologics lack specificity, activate both engineered and endogenous immune cells, and exhibit toxicity in healthy tissue which limits maximum tolerable doses and narrows their therapeutic windows. Thus, what is needed are CAR T cells with locally augmented functions at tumor and disease sites such as draining lymph nodes thereby improving the safety and efficacy of cell-based therapies.

### SUMMARY

The present invention relates to heat activated promoter constructs and methods for the manufacture and use thereof.

In one aspect, disclosed herein are promoter constructs comprising a) one or more heat shock elements (such as, for example, the heat shock element as set forth in SEQ ID NOs: 1, 2, 3, 4, 5, 6, 7, 8, and/or 9); b) a core promoter; and c) a gene of interest.

Also disclosed are promoter constructs of any preceding aspect, wherein said promoter requires thermal activation between 40° C.-45° C. (such as, for example, between 40° C. and 42° C. or between 41° C. and 43° C. or between 42° C. and 45° C., including, but not limited to 40.0, 40.1, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some embodiments the promoter requires a thermal activation of at least 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.).

In some aspects, disclosed herein are promoter constructs of any preceding aspect, wherein the heat shock element is repeated at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 times. For example, in some aspects the heat shock element comprises seven repeats of SEQ ID NO:1.

Also disclosed herein are promoter constructs of any preceding aspect, wherein the core promoter comprises a heat shock protein core promoter (including, but not limited to the core promoter of heat shock protein HSPA1A, HSPH1, HSPB1, HSPA6, or YB such as, for example a heat shock protein core promoter comprising any one of the following nucleotide sequences SEQ ID NOS: 10-13).

In some aspects, disclosed herein are promoter constructs of any preceding aspect, wherein the gene of interest encodes any combination of the following: a) a reporter protein (such as, for example, luciferase, green fluorescent protein (GFP), yellow fluorescent protein (YFP), blue fluorescent protein (BFP), cyane fluorescent protein (CFP), monomeric red fluorescent protein (mRFP), Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2; b) an immunomodulating agent (such as, for example, chemokines (including, but not limited to CCL2, CCL1, CCL19, CCL22, CXCL12, CCL17, MIP-1α, MCP-1, GRO/KG, and/or CXCR3) cytokines (including, but not limited to IL-1β, IL-2, IL-4, IL-6, IL-8, IL-10, IL-12, IL-15, IL-18, IL-21, IL-22, IFN-γ, TNF-α, TGF-β, LIF, and/or cytotoxins (including, but not limited to perforin and/or granzyme); c) a bispecific T cell engager antibody including, but not limited to a bispecific T cell engager antibody comprising an anti-CD-3 binding domain and an NKG2D receptor extracellular domain; d) a chimeric antigen receptor (CAR)(including, but not limited to a CAR targeting CD19, B cell maturation antigen (BCMA), CD22, CD33, CD38, NCAMI, CD5, CD70, MET, Muc1, LICAM, CD44 SLAMF7, EGFR, EPHA2, GPC3, HER2, mesothelin, or PDCD1); and/or e) a recombinant T cell receptor (TCR)(including, but not limited to a TCR targeting WT1, HPV E6, HPV E7, NY-ESO-1, HA-1, MAGE, Gp100, MART-1, HBV, p53, CEA. SL9, TGFβ11, TRAIL, MCPyV, PRAME, EBV, CMV, or KRAS.

Also disclosed herein are kits comprising the promoter constructs of any preceding aspect and further comprising a heating element to activate the promoter construct. In some aspects, the heating element can be a light source (such as for example, a laser (including, but not limited a near infrared laser), filament, infrared emitting light source, or light emitting diode (LED)), thermal pad, or thermally regulated needle, probe, or scalpel).

In some aspects, disclosed herein are immune cells comprising the promoter construct of any preceding aspect. In some embodiments, the immune cell is a T cell, natural killer (NK) cell, or dendritic cell. In some embodiments, the T cell comprises a recombinant TCR. In some embodiments, the immune cell is a chimeric antigen receptor (CAR) T cell and/or CAR natural killer (NK) cell. For example, disclosed herein are CAR T or CAR NK cells comprising a promoter construct comprising a) one or more heat shock elements (such as, for example, the heat shock element as set forth in SEQ ID NO: 1); b) a core promoter; and c) a gene of interest. In some embodiments, the gene of interest encodes a chimeric antigen receptor, a recombinant TCR, an immunomodulating agent, or any combination thereof.

Also disclosed herein are methods of treating, reducing, decreasing, inhibiting, ameliorating, and/or preventing a cancer and/or metastasis (such as for example, a solid tumor including, but not limited to, epithelial carcinoma, a sarcoma, a lymphoma, a blastoma, or a melanoma) in a subject comprising administering to the subject the promoter, the immune cell, T cell (e.g., CAR T cell), NK cell (e.g., CAR NK cell), or dendritic cell, or applying the kit any preceding aspect. For example, disclosed herein are methods of treating, reducing, decreasing, inhibiting, ameliorating, and/or preventing a cancer and/or metastasis (such as for example, a solid tumor including, but not limited to, epithelial carcinoma, a sarcoma, a lymphoma, a blastoma, or a melanoma) in a subject comprising administering to the subject a thermally controlled CAR immune cell (such as, for example, a CAR T cell or CAR NK cell comprising a promoter construct comprising a) one or more heat shock elements (such as, for example, the heat shock element as set forth in SEQ ID NO: 1); b) a core promoter; and c) a gene of interest) and inducing activation of the CAR T cell and/or CAR NK cell at the site of the tumor (such as, for example, heating the CAR T cell and/or CAR NK cells to between 40° C.-45° C. (such as, for example, between 40° C. and 42° C. or between 41° C. and 43° C. or between 42° C. and 45° C., including, but not limited to 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some embodiments the promoter requires a thermal activation of at least 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some aspects, the method can further comprise administering an additional anticancer agent or immunotherapy (including, but not limited checkpoint inhibitor such as used in anti-PD-1 immunotherapy, anti-PD-L1 immunotherapy, anti-CTLA-4 immunotherapy).

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIGS. 1A-1I show construction and activity of thermal-specific gene switches (FIG. 1A) Schematic of a panel of six thermal gene switch constructs comprising 2 to 7 heat shock elements (HSEs) upstream of the HSPB1 core promoter (labeled 2H-B1 to 7H-B1). Capitalized base pairs within HSE were conserved while base pairs indicated as n were randomized. (FIG. 1B) Gluc reporter expression by Jurkat T cells following heating as a function of HSE number or (FIG. 1C) temperature (ns=not significant, *P<0.05, **P<0.01, two-way ANOVA and Tukey post-test and correction, mean±SEM is depicted, n=3 biologically independent wells). Three independent experiments were performed with similar results. (FIG. 1D) Kinetics of Gluc reporter expression by primary human T cells following heat treatments at indicated temperatures (****P<0.0001, two-way ANOVA and Tukey post-test and correction, mean±SEM is depicted, n=3 biologically independent wells). Two independent experiments were performed with similar results. (FIG. 1E) Activity of thermal gene switches containing different core promoter constructs following heat treatments in primary human T cells (ns=not significant, *P<0.05, one-way ANOVA and Tukey post-test and correction, mean±SEM is depicted, n=3 biologically independent wells). Two independent experiments were performed with similar results. (FIG. 1F) Gluc expression in primary human T cells that were incubated 24 hours at the displayed temperatures. n=3. (FIG. 1G) Activity of 7H-YB in primary human T cells from multiple human donors following 30 minutes of heat treatment at the indicated temperatures (****P<0.0001, student t-test, mean±SEM is depicted, n=3 biologically independent wells). (FIG. 1H) Activity of 7H-YB compared to endogenous HSP70 and HSPA6 promoters in primary human T cells following exposure to CoCl₂ to mimic hypoxia or (FIG. 1I) to CdCl₂ to model heavy metal toxicity (ns=not significant, *P<0.05, ****P<0.0001, two-way ANOVA and Tukey post-test and correction, mean±SEM is depicted, n=3 biologically independent wells).

FIG. 2 shows the qPCR screen of HSPs in primary murine T cells. Splenic CD8+ T cells were isolated using the CD8+ T cell isolation kit according to (Miltenyi 130-104-075). Six hours after indicated heat treatments, mRNA was harvested and quantified using the Mouse HSP profiler kit (Qiagen PAMM-076Z) according to manufacturer instructions. Data are displayed relative to unheated controls.

FIG. 3 shows the transduction efficiencies of primary human T cells from three donors. Flow cytometric plots of primary human T cells derived from 3 donors and transduced with the Gluc expressing 7H-YB Thermal switch containing a constitutively expressed mCherry reporter. Inset shows the mean fluorescent intensity (MFI) of mCherry transduced cells.

FIGS. 4A-4B show the thermal switch specificity in Jurkat T cells. Gluc activity by Jurkat T cells transduced with synthetic thermal gene switch constructs (blue) or the endogenous HSPA6 promoter (red) following exposure to (FIG. 4A) CoCl₂ to mimic hypoxia or (FIG. 4B) to CdCl₂ to model heavy metal toxicity (ns=not significant, *P<0.05, ****P<. 0001, two-way ANOVA and Tukey post-test and correction, error bars show SEM, n=3).

FIGS. 5A-5E show thermal treatments are well-tolerated by primary human T cells. (FIG. 5A) Gluc activity of the 7H-YB thermal switch in primary human T cells after continuous (light grey) and pulsed (dark grey) heat treatments with temperatures, total durations, and heating profiles as indicated (ns=not significant, *P<0.05, **P<0.01, ****P<0.0001, two-tailed t-test, mean±SEM is depicted, n=3 biologically independent wells). (FIG. 5B) Propidium Iodide (PI) and Annexin V flow staining of CD3+ T cells. Bars represent viable populations (PI⁻Annexin V⁻) normalized to unheated samples (ns=not significant, ****P<0.0001, one-way ANOVA and Dunnett post-test and correction, mean±SEM is depicted, n=3 biologically independent wells). Two independent experiments were performed with similar results. (FIG. 5C) CellTrace Violet (CTV) flow histograms of T cells after heat treatments and incubation with CD3/28 beads at a 3:1 bead to T cell ratio. Two independent experiments were performed with similar results. (FIG. 5D) Number of cells in lower well of a transwell plate containing CXCL12. T cells were heated and loaded into the top well prior to sampling at indicated timepoints (ns=not significant between 37° C. and 42° C., two-way ANOVA and Tukey post-test and correction, mean±SEM is depicted, n=3 biologically independent wells). Two independent experiments were performed with similar results. (FIG. 5E) Percent cytotoxicity observed in CD19− or CD19+ luciferized K562 cells after incubation with T cells constitutively expressing CARs after heating with effector to target ratios as indicated (ns=not significant, *P<0.05, two-way ANOVA and Sidak post-test and correction, mean±SEM is depicted, n=3 biologically independent wells). Two independent experiments were performed with similar results.

FIG. 6 shows the gating strategy for viability flow staining. Primary human T cells were heated at 42° C. for 60 minutes as a positive control for thermal damage. Shorter regimens were used for subsequent experiments. Because many of the AnnexinV+ or PI+ events were not within tighter FSC/SSC gates, this conservative gating strategy was used as it better represented the sample's overall viability.

FIG. 7 shows the longitudinal heating of primary human T cells. Primary human T cells transduced with an HSPA6-GFP switch were repeatedly heated once GFP signal had returned to baseline after previous heat treatment (n=3 biologically independent wells, error bars show SEM). Two independent experiments were performed with similar results.

FIGS. 8A-8B show the repeated heat treatments do not affect CAR T cell cytotoxicity. (FIG. 8A) Primary human T cells were transduced to constitutively express an αCD19 CAR following CD3/CD28 bead activation. Heat treatments were performed at indicated timepoints prior to coincubation with luciferized, CD19+ K562s according to the timeline. (FIG. 8B) Percent cytotoxicity was quantified by loss of luminescence in wells relative to control wells containing only target cells (b) (ns=not significant, two-way ANOVA and Sidak post-test and correction, mean±SEM is depicted, n=3 biologically independent wells).

FIGS. 9A-9F show the photothermal activation of engineered T cells in vivo. (FIG. 9A) Thermal and luminescent images of wells containing TS-Fluc T cells after irradiation with NIR laser light. Thermal images (left) were acquired using a FLIR thermal camera while luminescent images (right) were acquired using an IVIS Spectrum CT system 6 hours after heat. (FIG. 9B) Schematic representation of TS-Fluc αCD19 CAR construct transduced into primary human T cells before transfer into NSG mice with two flank (K562 or Raji) tumors followed by photothermal heating of single tumor. (FIG. 9C) Thermal images of mouse during laser irradiation of tumor site at 0 and 3 minutes. (FIG. 9D) Kinetic traces (colored lines) showing average skin temperature of a 3×3 pixel ROI centered on laser site. Shaded regions show standard deviation of 3 heating runs. (FIG. 9E) Left: Luminescent images of heated mice bearing either K562 (CD19−) or Raji (CD19+) tumors. Signal indicates luciferase activity by transferred TS-Fluc T cells. Right: Luminescence of each tumor site relative to the luminescence from the unheated tumor in the same animal. ROIs were drawn as indicated in left panel. A separate experiment with repeated heating of Raji tumors was conducted to confirm reproducibility of experimental results (FIG. 11). (FIG. 9F) Mice bearing two Raji (CD19+) tumors with one site heated. Left: luminescent images of excised tumors (heated and unheated) and spleen. Right: quantification of luminescence following heat treatments (0, 6, 12, 18, 24 hrs). ns=not significant, ****P<0.0001, two-way ANOVA and Tukey post-test and correction, mean±SEM is depicted, n=4-5 biologically independent mice.

FIG. 10 shows CD19 expression on K562 and Raji tumor cells. Representative flow cytometric plots of CD19 staining on K562 and Raji cell lines (Iso=isotype control).

FIG. 11 shows the longitudinal control of intratumoral CAR T cells using photothermal pulses. Mice bearing Raji tumors (CD19+) were injected i.v. with TS-Fluc T cells. Tumor sites were irradiated on days 2 and 4 using NIR laser light as shown in FIG. 18d. Luminescence was quantified daily via i.v. injections of D-luciferin (n=3, biologically independent wells, error bars show SEM).

FIG. 12 shows the TS-Fluc αCD19 CAR T cell infiltration into K562 and Raji flank tumors. TS-Fluc αCD19 CAR T cells were injected i.v. into tumor bearing mice once tumors had reached ˜250 mm³. After 7 days, tumors were resected, dissociated, and stained to quantify cellular infiltration using flow cytometry counting beads (n=3, biologically independent wells, error bars show SEM).

FIGS. 13A-13I show the photothermal control of IL-15 SA enhances adoptive T cell transfer and overall survival in mice. (FIG. 13A) Schematic of co-culture assay of heated TS-IL15 αCD19 cells and CFSE-labeled wild-type cells. CD3/28 beads were added at 1:10 bead to T cell ratio. (FIG. 13B) Representative flow histograms (left), quantified proliferation (middle), and division indices (right) as calculated by FlowJo proliferation tool of the CFSE-labeled wild-type T cell population after thirty minute thermal treatment at indicated temperatures (*P<0.05, two-tailed t-test, mean±SEM is depicted, n=3 biologically independent wells). (FIG. 13C) IL-15 superagonist concentrations in supernatant of TS-IL15 αCD19 T cells following heat treatments. Temperature and duration of treatments are as indicated (ns=not significant, **P<0.01, ****P<0.0001, two-way ANOVA and Tukey post-test and correction, mean±SEM is depicted, n=3 biologically independent wells, comparisons are to unheated control). Two independent experiments were performed with similar results. (FIG. 13D) Schematic of TS-IL15 αCD19 CAR vector used to transduce primary human T cells before transfer into tumor bearing NSG mice. (FIG. 13E) Tumor growth curves of CD19+ K562s following transfer of TS-IL15 αCD19 CAR T cells on day 0 and heat treatments on days 2, 6, 9, 13, and 16 (ns=not significant, *P<0.05, **P<0.01, ***P<0.001, ****P<0.0001, two-way ANOVA and Tukey post-test and correction, mean±SEM is depicted, n=7 biologically independent mice). Three independent experiments were performed with similar results. (FIG. 13F) Survival curves of tumor-bearing mice in (FIG. 13D) and (FIG. 13E) following transfer of TS-15 αCD19 CAR T cells and heat treatments (**p<0.01, Log-rank (Mantel-Cox) test with correction for 6 multiple comparisons, n=7 biologically independent mice). (FIG. 13G) Schematic of TS-IL15 vector transduced into primary murine Pmel-1 T cells transferred into tumor bearing C57BL/6J mice. (FIG. 13H) Tumor growth curves following inoculation of B16-F10 following transfer of TS-IL15 Pmel-1 T cells on day 0 and heat treatments on days 1 and 3 (ns=not significant, ***P<0.001, two-way ANOVA and Tukey post-test and correction, mean±SEM is depicted, n=6-7 biologically independent mice). (FIG. 13I) Survival curves of tumor-bearing mice in (FIG. 13G) and (FIG. 13H) following transfer of TS-15 Pmel-1 T cells and heat treatments (**p<0.01, Log-rank (Mantel-Cox) test with correction for 6 multiple comparisons, n=7 biologically independent mice).

FIG. 14 shows cytokine support improves proliferation of T cells receiving low levels of CD3/28 stimulation. T cells were labeled with CFSE and incubated with low levels of activating beads. For reference, routine expansion and culture of T cells uses 3 beads for every T cell. Increasing amounts of IL-2 were added to each bead ratio. All samples were assayed after 4 day incubations at indicated conditions. Two independent experiments were performed with similar results.

FIG. 15 shows the gating strategy for mixed proliferation experiment. Transduced TS-15 αCD19 CAR T cells were identified by CAR expression. Proliferation of CFSE+ wild-type cells was assessed by dye dilution and FlowJo proliferation tool.

FIGS. 16A-16B show the characterization of engineered Pmel-1 T cells. 48 hours post isolation and peptide activation, Pmel-1 derived splenocytes were transduced with the TS-IL15 vector containing a constitutive GFP reporter. (FIG. 16A) Pmel-1 T cells were characterized via flow cytometry before adoptive transfer to assess CD8+ cell purity expansion (left) and transduction efficiency (right) (U.S.=Unstained Pmel-1 T cells, W.T=Wild type Pmel-1 T cells). (FIG. 16B) IL-15 production measured via ELISA from transduced murine T cells after 20 minute heating at indicated temperatures (***P<0.001, unpaired T test, mean±SEM is depicted, n=3 biologically independent wells).

FIGS. 17A-18B show that engineered Pmel-1 T cells enhance adoptive cell therapy in a high tumor burden setting. (FIG. 17A) Schematic representation of large tumor B16-F10 bearing C57BL/6J mice. (FIG. 17B) Tumor growth curves following inoculation of B16F10 following transfer of engineered murine T cells on day 0 and heat treatments on Days 1, 3, and 5 (*P<0.05, **P<0.01, two-way ANOVA and Tukey post-test and correction, mean±SEM is depicted, n=6-7 biologically independent mice).

FIGS. 18A-18I show expanding CAR T cell targeting via heat-triggered BiTEs. (FIG. 18A) Schematic of TS-BiTE and TS-Fluc thermal switches containing heat-triggered BiTE or Fluc reporters. Both constructs contained constitutive αCD19 CARs. (FIG. 18B) Histograms for HisTag flow staining in TS-BiTE and TS-Fluc primary T cells following heating. (FIG. 18C) NKG2DL flow staining on primary human T cells and K562s using an NKG2D-Fc chimera and an αFc-A488 secondary antibody. Stain=full staining, 2° Ctrl=secondary antibody only. (FIG. 18D) Schematic depicting BiTE-mediated targeting of K562 target cells lacking the CAR target antigen via BiTE binding to NKG2DL and CD3. (FIG. 18E) Flow gating strategy for defining bystander cells based on CD19 CAR expression in Jurkat co-culture assays with K562s. UTD controls were gated on the lower (CAR−) population for graphing in (FIG. 18G). (FIG. 18F) Flow staining of CD69 on Jurkat T cells following heating and incubation with K562s. TS-BiTE CAR+ histograms (f) and summary data of indicated populations (FIG. 18G) are graphed (stats show comparison to UTD, ns=not significant, **P<0.01, ****P<0.0001, two-way ANOVA and Tukey post-test and correction, mean±SEM is depicted, n=3 biologically independent wells). Two independent experiments were performed with similar results. (FIG. 18H) Cytokine concentrations in supernatant of primary human T cells after heat treatments and incubation with K562 cells. T cells were either untransduced or transduced with the indicated thermal switches (ns=not significant, ****P<0.0001, two-way ANOVA and Tukey post-test and correction, mean±SEM is depicted, n=3 biologically independent wells). (FIG. 18I) Cytotoxicity against K562s as quantified by luciferase assay after incubation with primary human T cells. T cells were either untransduced or transduced with the indicated thermal switches (ns=not significant, **P<0.01, ****P<0.0001, two-way ANOVA and Tukey post-test and correction, comparisons are to UTD control, mean±SEM is depicted, n=3 biologically independent wells.

FIGS. 19A-19F show the photothermal control of TS-BiTE αHER2 CAR T cells mitigates outgrowth of antigen negative tumors in vivo. (FIG. 19A) Schematic of TS-BiTE and TS-Rluc αHER2 CAR vectors. (FIG. 19B) Flow cytometry quantification of activation markers CD69, PD-1 and CD107a by TS-BiTE αHER2 CAR T cells after 30-minute heating and coculture with HER2-MDA-MB-468 target cells. (ns=not significant, ****P<0.0001, two-way ANOVA and Tukey post-test and correction, error bars show SEM, n=4 biologically independent wells, MFI=Median fluorescent intensity). (FIG. 19C) Tumor growth curves of MDA-MB-468 tumors inoculated at a 3:1 HER2+ to HER2-ratio treated with TS-BiTE or TS-Rluc αHER2 CAR T cells. Heat treatments were carried out on days 45, 47, 52, 59, 66, and 72. (*P<0.05, ****P<0.0001, two-way ANOVA and Tukey post-test and correction, mean±SEM is depicted, n=6-7 biologically independent mice). (FIG. 19D) Spider plots of individual tumors with vertical dashed lines indicating heat treatments. (FIG. 19E) In vivo luminescent imaging time course and (FIG. 19F) individual spider plots acquired by an IVIS Spectrum CT system representative of the HER2−/Fluc+ cell population in the mixed MDA-MB-468 tumor model. Transfer of engineered human T cells occur on day 44 and heat treatments on days 45, 47, 52, 59, 66, and 72. Grey background represents mean±2 standard deviations above and below background measurements (n=6) taken throughout the timecourse of the experiment. (ns=not significant, *P<0.05, **P<0.01, ***P<0.001, ****P<0.0001, two-way ANOVA and Tukey post-test and correction, mean±SEM is depicted, n=6-7 biologically independent mice).

FIGS. 20A-20C shows the validation of MDA-MB-468 transduced with HER2 or Renilla Luciferase. (FIG. 20A) Representative flow plots of NKG2DL staining and HER2 Staining of HER2+ MDA-MB-468s. MDA-MB-468 were transduced with lentivirus to stably surface express HER2. (FIG. 20B) Luminescence of Fluc transduced HER2-MDA-MB-468 tumor cells (****P<0.0001, one-way ANOVA and Sidak post-test and correction, mean±SEM is depicted, n=3 biologically independent wells). (FIG. 20C) Percent cytotoxicity observed via LDH assay in HER2− or HER2+ MDA-MB-468 cells after incubation with T cells constitutively expressing CARs (****P<0.0001 between HER2− and HER2+ groups, two-way ANOVA and Sidak post-test correction, mean±SEM is depicted, n=3 biologically independent wells). Two independent experiments were performed with similar results.

FIG. 21 shows that TS-BiTE αHER2 CAR T cells activate when incubated with HER2+ MDA-MB-468 cells. MFIs of activation and degranulation markers CD69, PD-1, and CD107a on TS-BiTE αHER2 CAR T cells that were heated at indicated temperatures prior to co-incubation with HER2+ MDA-MB-468 target cells. (ns=not significant, ****P<0.0001, two-way ANOVA and Tukey post-test and correction, error bars show SEM, n=4 biologically independent wells).

FIG. 22A shows a schematic of I.T. injection of IL-2 in B16 tumor.

FIG. 22B shows tumor growth curves following ACT of Pmel CD8+ T cells. Six i.p. hIL-2 injections were administered at indicated doses following ACT.

FIG. 23 shows dendritic cells engineered with TS-IL15SA produce IL-15SA upon thermal treatment at 42° C. for 30 min.

### DETAILED DESCRIPTION

The following description of the disclosure is provided as an enabling teaching of the disclosure in its best, currently known embodiment. To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Definitions

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity.

Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular promoter construct or modified CAR T cell is disclosed and discussed and a number of modifications that can be made to a number of molecules including the promoter construct or modified CAR T cell are discussed, specifically contemplated is each and every combination and permutation of promoter construct or modified CAR T cell and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D. C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including matters of logic with respect to arrangement of steps or operational flow; plain meaning derived from grammatical organization or punctuation; and the number or type of embodiments described in the specification.

Emerging strategies to control engineered T cells and augment their anti-tumor activity include the use of biomaterials to co-deliver adjuvants to the TME as well as genetic constructs for autonomous expression of immunostimulatory genes. For example, implantation of biopolymer scaffolds loaded with tumor-specific T cells and immunostimulatory adjuvants at the surgical site improved postoperative responses following primary tumor resection in mouse models. To provide a localized source of adjuvants, T cells tethered on their cell surface to nanoparticle ‘backpacks’ allowed infiltrating T cells to carry cargo and release a one-time dose of drug within tumors. Increasingly sophisticated genetic circuitry has also allowed T cells to locally produce biologics to overcome immunosuppression or target antigens after tumor infiltration. For example, ‘armored CARs’ leverage constitutive expression of biologics such as IL-12, αPD-1 scFvs, and BiTEs to improve anti-tumor activity. T cells have also been engineered with sense- and respond biocircuits that conditionally activate in the presence of specific input signals. These strategies include NFAT-inducible cassettes that upregulate expression of cytokines following T cell recognition of a tumor-associated antigen. To further increase specificity, T cells have been engineered to target unique combinations of epitopes expressed in the TME to allow discrimination from healthy cells expressing a single epitope. Such approaches based on Boolean logic require the presence of both target antigens for T cell activation to occur and have demonstrated efficacy in multiple models of focal tumors. Collectively, these approaches illustrate the need to develop strategies to control and improve intratumoral T cell activity.

Thus, in one aspect, disclosed herein are promoter constructs comprising a) one or more heat shock elements (such as, for example, the heat shock element as set forth in SEQ ID NO: 1); b) a core promoter; and c) a gene of interest.

Heat shock elements are cis acting regulator motifs that mediate transcriptional response of target genes when exposed to heat. One example of a heats shock element is nGAAnnTTCnnGAAn (SEQ ID NO: 1). In some embodiments, n=A, T, C, or G. The heat shock element can be repeated at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 times. For example, in some aspects the heat shock element comprises seven repeats of SEQ ID NO:1. Examples of sequences for one or more heat shock elements are provided in Table 1. In some embodiments, the one or more heat shock elements comprises or consists of the nucleotide sequence of any one of SEQ ID NOS:2-9.

TABLE 1
Heat Shock Element Sequences

| | | SEQ |
|---|---|---|
| | | ID |
| Description | Sequence | NO: |
| 1 HSE | Agaacgttctagaacttgc | 2 |
| 2 HSEs | agaacgttctagaaggtctagaacg | 3 |
| | ttctagaacttgc | |
| 3 HSEs | tgaaagttctagaacgacgagaacg | 4 |
| | ttctagaaggtctagaacgttctag | |
| | aacttgc | |
| 4 HSEs | agaagcttctagaatgtgctgaaag | 5 |
| | ttctagaacgacgagaacgttctag | |
| | aaggtctagaacgttctag | |
| | aacttgc | |
| 5 HSEs | agaacgttctagaacctggagaagc | 6 |
| | ttctagaatgtgctgaaagttctag | |
| | aacgacgagaacgttctagaaggtc | |
| | tagaacgttctagaacttgc | |
| 6 HSEs | Agaacgttcatgaacgctgagaacg | 7 |
| | ttctagaacctggagaagcttctag | |
| | aatgtgctgaaagttctagaacgac | |
| | gagaacgttctagaaggtctagaac | |
| | gttctagaacttgc | |
| 7 HSEs | agaagcttcatgaacgtgcagaacg | 8 |
| | ttcatgaacgctgagaacgttctag | |
| | aacctggagaagcttctagaatgtg | |
| | ctgaaagttctagaacgacgagaac | |
| | gttctagaaggtctagaacgttcta | |
| | gaacttgc | |
| 8 HSEs | agaagcttcatgaacgtgcagaagc | 9 |
| | ttcatgaacgtgcagaacgttcatg | |
| | aacgctgagaacgttctagaacctg | |
| | gagaagcttctagaatgtgctgaaa | |
| | gttctagaacgacgagaacgttcta | |
| | gaaggtctagaacgttctagaactt | |
| | gc | |

In some aspect, the core promoter comprises a heat shock protein transcription start site. Such heat shock protein transcription start site are known in the art and can include but are not limited to heat shock protein transcription start site of HSPA1A, HSPH1, HSPB1, HSP6, HSP70, HSPA6, or YB. In some embodiments, the core promoter comprises the core promoter of heat shock protein HSPA1A, HSPH1, HSPB1, HSP6, HSP70, HSPA6, or YB. Examples of core promoter sequences are provided in Table 2. In some embodiments, the core promoter sequence comprises or consists of any one of SEQ ID NOS:10-13.

TABLE 2
Core Promoter Sequences

| | | SEQ |
|---|---|---|
| | | ID |
| Name | Sequence | NO: |
| HSPA1A | ttaaaggcgcagggcggcgagcagg | 10 |
| Core | tcaccagacgctgacagctactcag | |
| Promoter | aaccaaatctggttccatccagaga | |
| | caagcgaagacaagagaagcagagc | |
| | gagcggcgcgttcccgatcctcggc | |
| | caggaccagccttccccagagcatc | |
| | cctgccgcggagcgcaaccttccca | |
| | ggagcatccctgccgcggagcgcaa | |
| | ctttccccggagcatccacgccgcg | |
| | gagcacagccttccagaagcagagc | |
| | gcggcgccctcgag | |
| HSPB1 | ttgccattaatagagacctgaagca | 11 |
| Core | ccgcctgctaaaaatacccggctgg | |
| Promoter | gcacacataaaagcacgctggggct | |
| | ccagtccggcacttctcggatcctc | |
| | agcccagtgcttctagatcctcagc | |
| | cttgaccagccaagaacatgac | |
| HSPA6 | taaaaagcccgtggaagcggagctg | 12 |
| Core | agcagatccgagccgggctggctgc | |
| Promoter | agagaaaccgcagggagagcctcac | |
| | tgctgagcgcccctcgacggcggag | |
| | cggcagcagcctccgtggcctccag | |
| | catccgacaagaagcttcagccacc | |
| | ggtctcgag | |
| YB Core | tctagagggtatataatgggggcca | 13 |
| Promoter | ctagtctactaccagaaagcttggt | |
| | accgagctcggatccagccacc | |

Examples of one or more heat shock element sequence and YB core promoter sequence together are provided in Table 3. In some embodiments, the one or more heat shock elements and core promoter together comprise a sequence set forth in any one of SEQ ID NOS:14-21.

TABLE 3
Heat Shock Element + Core
Promoter Sequences

| | | SEQ |
|---|---|---|
| | | ID |
| Description | Sequence | NO: |
| 1 HSE + YB | Agaacgttctagaacttgctctaga | 14 |
| core promoter | gggtatataatgggggccactagtc | |
| | tactaccagaaagcttggtaccgag | |
| | ctcggatccagccacc | |
| 2 HSEs + YB | agaacgttctagaaggtctagaacg | 15 |
| core promoter | ttctagaacttgctctagagggtat | |
| | ataatgggggccactagtctactac | |
| | cagaaagcttggtaccgagctcgga | |
| | tccagccacc | |
| 3 HSEs + YB | tgaaagttctagaacgacgagaacg | 16 |
| core promoter | ttctagaaggtctagaacgttctag | |
| | aacttgctctagagggtatataatg | |
| | ggggccactagtctactaccagaaa | |
| | gcttggtaccgagctcggatccagc | |
| | cacc | |
| 4 HSEs + YB | agaagcttctagaatgtgctgaaag | 17 |
| core promoter | ttctagaacgacgagaacgttctag | |
| | aaggtctagaacgttctagaacttg | |
| | ctctagagggtatataatgggggcc | |
| | actagtctactaccagaaagcttgg | |
| | taccgagctcggatccagccacc | |
| 5 HSEs + YB | agaacgttctagaacctggagaagc | 18 |
| core promoter | ttctagaatgtgctgaaagttctag | |
| | aacgacgagaacgttctagaaggtc | |
| | tagaacgttctagaacttgctctag | |
| | agggtatataatgggggccactagt | |
| | ctactaccagaaagcttggtaccga | |
| | gctcggatccagccacc | |
| 6 HSEs + YB | Agaacgttcatgaacgctgagaacg | 19 |
| core promoter | ttctagaacctggagaagcttctag | |
| | aatgtgctgaaagttctagaacgac | |
| | gagaacgttctagaaggtctagaac | |
| | gttctagaacttgctctagagggta | |
| | tataatgggggccactagtctacta | |
| | ccagaaagcttggtaccgagctcgg | |
| | atccagccacc | |
| 7 HSEs + YB | agaagcttcatgaacgtgcagaacg | 20 |
| core promoter | ttcatgaacgctgagaacgttctag | |
| | aacctggagaagcttctagaatgtg | |
| | ctgaaagttctagaacgacgagaac | |
| | gttctagaaggtctagaacgttcta | |
| | gaacttgctctagagggtatataat | |
| | gggggccactagtctactaccagaa | |
| | agcttggtaccgagctcggatccag | |
| | ccacc | |
| 8 HSEs + YB | agaagcttcatgaacgtgcagaagc | 21 |
| core promoter | ttcatgaacgtgcagaacgttcatg | |
| | aacgctgagaacgttctagaacctg | |
| | gagaagcttctagaatgtgctgaaa | |
| | gttctagaacgacgagaacgttcta | |
| | gaaggtctagaacgttctagaactt | |
| | gctctagagggtatataatgggggc | |
| | cactagtctactaccagaaagcttg | |
| | gtaccgagctcggatccagccacc | |

The use of the heat shock element allows selective transcriptional control of the gene of interest such that the gene of interest is only activated once heat within a desired temperature range is applied. Thus, for example, disclosed herein are promoter constructs, wherein said promoter requires thermal activation between 40° C.-45° C. (such as, for example, between 40° C. and 42° C. or between 41° C. and 43° C. or between 42° C. and 45° C., including, but not limited to 40.0, 40.1, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some embodiments the promoter requires a thermal activation of at least 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.).

The gene of interest used in the disclosed constructs can be a reporter gene, an immunomodulating agent, a bispecific T cell engager (BiTE), a chimeric antigen receptor (CAR), a recombinant TCR, or any combination thereof.

A BiTE refers to a bispecific fusion protein refers to a single chain protein composed of two linked scFvs, one of which targets a T cell (CD3) and the other targets a tumor cell antigen. Examples of BiTE molecules include those comprising an anti-CD3 binding domain and a NKG2D receptor extracellular domain, an anti-CD3 binding domain and an anti-EGFRvIII binding domain, and an anti-CD3 binding domain and an anti-CD19 binding domain.

Reporter genes are well known in the art and can include any gene whose transcription and/or translation can be readily assayed subsequent to transfection. Examples of reporter genes for used in the disclosed promoter constructs include for example, luciferase, green fluorescent protein (GFP), yellow fluorescent protein (YFP), blue fluorescent protein (BFP), cyane fluorescent protein (CFP), monomeric red fluorescent protein (mRFP), Discosoma striata (DsRed), mCherry, mOrange, tdTomato, mSTrawberry, mPlum, photoactivatable GFP (PA-GFP), Venus, Kaede, monomeric kusabira orange (mKO), Dronpa, enhanced CFP (ECFP), Emerald, Cyan fluorescent protein for energy transfer (CyPet), super CFP (SCFP), Cerulean, photoswitchable CFP (PS-CFP2), photoactivatable RFP1 (PA-RFP1), photoactivatable mCherry (PA-mCherry), monomeric teal fluorescent protein (mTFP1), Eos fluorescent protein (EosFP), Dendra, TagBFP, TagRFP, enhanced YFP (EYFP), Topaz, Citrine, yellow fluorescent protein for energy transfer (YPet), super YFP (SYFP), enhanced GFP (EGFP), Superfolder GFP, T-Sapphire, Fucci, mKO2, mOrange2, mApple, Sirius, Azurite, EBFP, and/or EBFP2.

In some aspects, the gene of interest can be an immunomodulating agent such as, for example, a chemokine, a cytokine, an interferon, a cytotoxin (including, but not limited to perforin and/or granzyme), or any combination thereof. Examples of chemokines that can be used in the disclosed promoter constructs include, but are not limited to CCL2, CCL1, CCL19, CCL22, CXCL12, CCL17, MIP-1α, MCP-1, GRO/KC, CXCL2, CXCR3, or any combination thereof. Cytokines that can be used in the disclosed promoter constructs include, but are not limited to IL-1β, IL-2, IL-4, IL-6, IL-8, IL-10, IL-12, IL-15, IL-18, IL-21, IL-22, IFN-γ, TNF-α, TGF-β, LIF, or any combination thereof. In some embodiments, the cytokine is an IL-15 super agonist molecule. An example of an IL-15 super agonist molecule is ALT-803, which is a multimeric complex composed of IL-15 N72D:IL-15Rα sushi domain fused to an IgG1 Fc domain.

Chimeric antigen receptors (CARs) are transgenic receptors expressed by a T cell (CAR T cell) or NK cell (CAR NK cell) that target the T cell or NK cell to cells expressing the ligand for the receptor. Such chimeric antigen receptors are typically membrane bound single chain variable regions of an immunoglobulin specific for the target. CAR targets include, but are not limited to CD19, B cell maturation antigen (BCMA), CD22, CD33, CD38, NCAMI, CD5, CD70, MET, Muc1, LICAM, CD44 SLAMF7, EGFR, EPHA2, HER2, mesothelin, GPC3, or PDCD1.

Recombinant T cell receptors (also referred to as engineered TCRs) refer to TCRs that are used to engineer T cells with a desired specificity, e.g., a tumor antigen. Examples of TCR targets include, but are not limited to WT1, HPV E6, HPV E7, NY-ESO-1, HA-1, MAGE, Gp100, MART-1, HBV, p53, CEA, SL9, TGFβ11, TRAIL, MCPyV, PRAME, EBV, CMV, and KRAS.

Promoter constructs of the disclosure may also include intervening nucleotides between the recited components. Junction nucleotides may be natural or non-natural (e.g., resulting from the construct design). For example, junction nucleotides may result from restriction enzyme sites used for joining one domain to another domain or cloning polynucleotides into vectors. Examples of promoter constructs of the present disclosure are provided in Table 4.

TABLE 4
Exemplary Promoter Constructs

| Name/ | |
|---|---|
| SEQ ID NO: | Sequence |
| Thermal gene | agaagcttcatgaacgtgcagaacgttcat |
| switch driving | gaacgctgagaacgttctagaacctggaga |
| expression of | agcttctagaatgtgctgaaagttctagaa |
| Firefly | cgacgagaacgttctagaaggtctagaacg |
| luciferase | ttctagaacttgctctagagggtatataat |
| (TS-Fluc)/ | gggggccactagtctactaccagaaagctt |
| SEQ ID NO: 22 | ggtaccgagctcggatccagccaccctcga |
| | gACCGGTatggaagacgccaaaaacataaa |
| | gaaaggcccggcgccattctatccgctgga |
| | agatggaaccgctggagagcaactgcataa |
| | ggctatgaagagatacgccctggttcctgg |
| | aacaattgcttttacagatgcacatatcga |
| | ggtggacatcacttacgctgagtacttcga |
| | aatgtccgttcggttggcagaagctatgaa |
| | acgatatgggctgaatacaaatcacagaat |
| | cgtcgtatgcagtgaaaactctcttcaatt |
| | ctttatgccggtgttgggcgcgttatttat |
| | cggagttgcagttgcgcccgcgaacgacat |
| | ttataatgaacgtgaattgctcaacagtat |
| | gggcatttcgcagcctaccgtggtgttcgt |
| | ttccaaaaaggggttgcaaaaaattttgaa |
| | cgtgcaaaaaaagctcccaatcatccaaaa |
| | aattattatcatggattctaaaacggatta |
| | ccagggatttcagtcgatgtacacgttcgt |
| | cacatctcatctacctcccggttttaatga |
| | atacgattttgtgccagagtccttcgatag |
| | ggacaagacaattgcactgatcatgaactc |
| | ctctggatctactggtctgcctaaaggtgt |
| | cgctctgcctcatagaactgcctgcgtgag |
| | attctcgcatgccagagatcctatttttgg |
| | caatcaaatcattccggatactgcgatttt |
| | aagtgttgttccattccatcacggttttgg |
| | aatgtttactacactcggatatttgatatg |
| | tggatttcgagtcgtcttaatgtatagatt |
| | tgaagaagagctgittctgaggagccttca |
| | ggattacaagattcaaagtgcgctgctggt |
| | gccaaccctattctccttcttcgccaaaag |
| | cactctgattgacaaatacgatttatctaa |
| | tttacacgaaattgcttctggtggcgctcc |
| | cctctctaaggaagtcggggaagcggttgc |
| | caagaggttccatctgccaggtatcaggca |
| | aggatatgggctcactgagactacatcagc |
| | tattctgattacacccgagggggatgataa |
| | accgggcgcggtcggtaaagttgttccatt |
| | ttttgaagcgaaggttgtggatctggatac |
| | cgggaaaacgctgggcgttaatcaaagagg |
| | cgaactgtgtgtgagaggtcctatgattat |
| | gtccggttatgtaaacaatccggaagcgac |
| | caacgccttgattgacaaggatggatggct |
| | acattctggagacatagcttactgggacga |
| | agacgaacacttcttcatcgttgaccgcct |
| | gaagtctctgattaagtacaaaggctatca |
| | ggtggctcccgctgaattggaatccatctt |
| | gctccaacaccccaacatcttcgacgcagg |
| | tgtcgcaggtcttcccgacgatgacgccgg |
| | tgaacttcccgccgccgttgttgttttgga |
| | gcacggaaagacgatgacggaaaaagagat |
| | cgtggattacgtcgccagtcaagtaacaac |
| | cgcgaaaaagttgcgcggaggagttgtgtt |
| | tgtggacgaagtaccgaaaggtcttaccgg |
| | aaaactcgacgcaagaaaaatcagagagat |
| | cctcataaaggccaagaagggcggaaagat |
| | cgccgtgtaa |
| Thermal gene | agaagcttcatgaacgtgcagaacgttcat |
| switch driving | gaacgctgagaacgttctagaacctggaga |
| expression of | agcttctagaatgtgctgaaagttctagaa |
| IL-15 SA | cgacgagaacgttctagaaggtctagaacg |
| (TS-IL15SA)/ | ttctagaacttgctctagagggtatataa |
| SEQ ID NO: 23 | tgggggccactagtctactaccagaaagct |
| | tggtaccgagctcggatccagccaccatg |
| | gcaccacgcagagccagaggttgccggact |
| | ctcggactgcccgcactgctgctcctcctg |
| | cttcttcggccgcctgccactagaggggac |
| | tacaaggacgacgatgacaagatcgaaggg |
| | aggattacgtgtcctcccccgatgtccgtg |
| | gaacacgcggacatctgggtcaagtcctat |
| | tccttgtactcccgcgagcggtacatttgc |
| | aactccggctttaagcgcaaagctggcacc |
| | agctccctcactgaatgcgtgctgaacaag |
| | gccactaatgtggcccattggaccaccccc |
| | tcgctgaagtgcatccgggaccctgccttg |
| | gtccaccaacgccctgcacctccatccgga |
| | ggatcaggcggaggaggttcgggtggtggt |
| | tccggtggaggagggagcctccagaactgg |
| | gtgaacgtgatcagcgaccttaagaaaatc |
| | gaggatctgattcagtcaatgcacatcgac |
| | gcgaccctctacaccgaaagcgacgtccac |
| | ccgagctgcaaggtcaccgccatgaagtgc |
| | ttcctgctggaactccaagtcatttcgctg |
| | gagagcggcgatgcttcaatccacgacact |
| | gtggaaaacctgatcattctggcaaacaac |
| | tccctctcttcgaatgggaacgtgaccgag |
| | tccggctgcaaggagtgcgaggagctggag |
| | gaaaagaacatcaaagagttcctgcagtcc |
| | ttcgtccacatcgtgcagatgttcatcaac |
| | acctcgtaa |
| Thermal gene | agaagcttcatgaacgtgcagaacgttcat |
| switch driving | gaacgctgagaacgttctagaacctggaga |
| expression of | agcttctagaatgtgctgaaagttctagaa |
| NKG2DL BITE | cgacgagaacgttctagaaggtctagaacg |
| (TS-BITE)/ | ttctagaacttgctctagagggtatataa |
| SEQ ID NO: 24 | tgggggccactagtctactaccagaaagct |
| | tggtaccgagctcggatccagccaccctc |
| | gagACCGGTatggagactgacaccctgctt |
| | ctctgggtgctcttgctttgggtgcctgga |
| | agcaccggcgaccaagtccaactgcaacag |
| | tcaggcgccgaactggctcggcctggagct |
| | tctgtgaagatgtcgtgcaaagcatccggc |
| | tacacctttactcgctacaccatgcactgg |
| | gtcaagcaaaggcccggacagggactggag |
| | tggattgggtacatcaacccttcgcggggg |
| | tacactaactacaaccagaagtttaaggac |
| | aaggccacgctgaccaccgacaagtcctcg |
| | tccactgcatacatgcagctctcctccctg |
| | acctccgaggactccgccgtgtactactgc |
| | gcccgctactacgacgaccactactgcctg |
| | gactactggggccagggtactaccctcacc |
| | gtgtcgtcaggaggcggaggaagcggtggc |
| | ggtggaagtggaggaggaggaagccagatc |
| | gtgctgactcagtccccggcgatcatgtcc |
| | gcgtcacctggcgaaaaggtcaccatgact |
| | tgtagcgcctcaagcagcgtgtcctacatg |
| | aactggtatcagcagaagtccggcacatcc |
| | cccaagcggtggatctatgacacttccaag |
| | ctggcctcaggagtgcctgcacatttccgc |
| | gggtctggttcgggcacctcctactccctg |
| | actatctcggggatggaagctgaggatgcg |
| | gccacctactactgccaacaatggtccagc |
| | aaccccttcaccttcgggagcggcactaag |
| | ctggaaatcaatgggggtggaggatcgggt |
| | ggaggcggatcaggagggggagggtcgttc |
| | ttgaatagcctgttcaaccaagaagtgcag |
| | atccccctgaccgaatcgtattgtggcccg |
| | tgcccaaagaactggatttgctacaagaac |
| | aactgctaccagttcttcgatgagtccaag |
| | aattggtacgagtcacaggcctcctgcatg |
| | agccagaacgcctccctcctgaaagtgtac |
| | tcgaaggaggaccaggatctgctgaagctg |
| | gtcaagtcctaccattggatgggcctggtg |
| | cacatcccgaccaacgggtcctggcagtgg |
| | gaggatgggtcgatcctgagccctaatctc |
| | ctcaccatcatcgagatgcagaagggagac |
| | tgcgccctgtacgcgagctcattcaagggc |
| | tacatagagaactgttcaactcccaacacc |
| | tacatctgcatgcagcggaccgtgcaccac |
| | caccatcaccactaa |
| Thermal gene | agaagcttcatgaacgtgcagaacgttcat |
| switch driving | gaacgctgagaacgttctagaacctggaga |
| expression of | agcttctagaatgtgctgaaagttctagaa |
| Renilla | cgacgagaacgttctagaaggtctagaacg |
| luciferase | ttctagaacttgctctagagggtatataa |
| (TS-Rluc)/ | tgggggccactagtctactaccagaaagct |
| SEQ ID NO: 25 | tggtaccgagctcggatccagccaccatg |
| | acttcgaaagtttatgatccagaacaaagg |
| | aaacggatgataactggtccgcagtggtgg |
| | gccagatgtaaacaaatgaatgttcttgat |
| | tcatttattaattattatgattcagaaaaa |
| | catgcagaaaatgctgttatttttttacat |
| | ggtaacgcggcctcttcttatttatggcga |
| | catgttgtgccacatattgagccagtagcg |
| | cggtgtattataccagaccttattggtatg |
| | ggcaaatcaggcaaatctggtaatggttct |
| | tataggttacttgatcattacaaatatctt |
| | actgcatggtttgaacttcttaatttacca |
| | aagaagatcatttttgtcggccatgattgg |
| | ggtgcttgtttggcatttcattatagctat |
| | gagcatcaagataagatcaaagcaatagtt |
| | cacgctgaaagtgtagtagatgtgattgaa |
| | tcatgggatgaatggcctgatattgaagaa |
| | gatattgcgttgatcaaatctgaagaagga |
| | gaaaaaatggttttggagaataacttcttc |
| | gtggaaaccatgttgccatcaaaaatcatg |
| | agaaagttagaaccagaagaatttgcagca |
| | tatcttgaaccattcaaagagaaaggtgaa |
| | gttcgtcgtccaacattatcatggcctcgt |
| | gaaatcccgttagtaaaaggtggtaaacct |
| | gacgttgtacaaattgttaggaattataat |
| | gcttatctacgtgcaagtgatgatttacca |
| | aaaatgtttattgaatcggacccaggattc |
| | ttttccaatgctattgttgaaggtgccaag |
| | aagtttcctaatactgaatttgtcaaagta |
| | aaaggtcttcatttttcgcaagaagatgca |
| | cctgatgaaatgggaaaatatatcaaatcg |
| | ttcgttgagcgagttctcaaaaatgaacaa |
| | taa |
| Thermal gene | agaagcttcatgaacgtgcagaacgttcat |
| switch driving | gaacgctgagaacgttctagaacctggaga |
| expression of | agcttctagaatgtgctgaaagttctagaa |
| Gaussia Luciferase | cgacgagaacgttctagaaggtctagaacg |
| (TS-Gluc)/ | ttctagaacttgctctagagggtatataa |
| SEQ ID NO: 26 | tgggggccactagtctactaccagaaagct |
| | tggtaccgagctcggatccagccaccctc |
| | gagACCGGTatgggagtcaaagttctgtt |
| | tgccctgatctgcatcgctgtggccgaggc |
| | caagcccaccgagaacaacgaagacttcaa |
| | catcgtggccgtggccagcaacttcgcgac |
| | cacggatctcgatgctgaccggggaagttg |
| | cccggcaagaagctgccgctggaggtgctc |
| | aaagagatggaagccaatgcccggaaagct |
| | ggctgcaccaggggctgtctgatctgcctg |
| | tcccacatcaagtgcacgcccaagatgaag |
| | aagttcatcccaggacgctgccacacctac |
| | gaaggcgacaaagagtccgcacagggcggc |
| | ataggcgaggcgatcgtcgacattcctgag |
| | attcctgggttcaaggacttggagcctatg |
| | gagcagttcatcgcacaggtcgatctgtgt |
| | gtggactgcacaactggctgcctcaaaggg |
| | cttgccaacgtgcagtgttctgacctgctc |
| | aagaagtggctgccgcaacgctgtgcgacc |
| | tttgccagcaagatccagggccaggtggac |
| | aagatcaagggggccggtggtgac |
| Thermal gene | agaagcttcatgaacgtgcagaacgttcat |
| switch driving | gaacgctgagaacgttctagaacctggaga |
| expression of | agcttctagaatgtgctgaaagttctagaa |
| a firefly | cgacgagaacgttctagaaggtctagaacg |
| luciferase | ttctagaacttgctctagagggtatataa |
| and membrane | tgggggccactagtctactaccagaaagct |
| bound RSV-F | tggtaccgagctcggatccagccaccctc |
| VHH (TS-VHH)/ | gagACCGGTatggaagacgccaaaaacat |
| SEQ ID NO: 27 | aaagaaaggcccggcgccattctatccgct |
| | ggaagatggaaccgctggagagcaactgca |
| | taaggctatgaagagatacgccctggttcc |
| | tggaacaattgcttttacagatgcacatat |
| | cgaggtggacatcacttacgctgagtactt |
| | cgaaatgtccgttcggttggcagaagctat |
| | gaaacgatatgggctgaatacaaatcacag |
| | aatcgtcgtatgcagtgaaaactctcttca |
| | attctttatgccggtgttgggcgcgttatt |
| | tatcggagttgcagttgcgcccgcgaacga |
| | catttataatgaacgtgaattgctcaacag |
| | tatgggcatttcgcagcctaccgtggtgtt |
| | cgtttccaaaaaggggttgcaaaaaatttt |
| | gaacgtgcaaaaaaagctcccaatcatcca |
| | aaaaattattatcatggattctaaaacgga |
| | ttaccagggatttcagtcgatgtacacgtt |
| | cgtcacatctcatctacctcccggttttaa |
| | tgaatacgattttgtgccagagtccttcga |
| | tagggacaagacaattgcactgatcatgaa |
| | ctcctctggatctactggtctgcctaaagg |
| | tgtcgctctgcctcatagaactgcctgcgt |
| | gagattctcgcatgccagagatcctatttt |
| | tggcaatcaaatcattccggatactgcgat |
| | tttaagtgttgttccattccatcacggttt |
| | tggaatgtttactacactcggatatttgat |
| | atgtggatttcgagtcgtcttaatgtatag |
| | atttgaagaagagctgtttctgaggagcct |
| | tcaggattacaagattcaaagtgcgctgct |
| | ggtgccaaccctattctccttcttcgccaa |
| | aagcactctgattgacaaatacgatttatc |
| | taatttacacgaaattgcttctggtggcgc |
| | tcccctctctaaggaagtcggggaagcggt |
| | tgccaagaggttccatctgccaggtatcag |
| | gcaaggatatgggctcactgagactacatc |
| | agctattctgattacacccgagggggatga |
| | taaaccgggcgcggtcggtaaagttgttcc |
| | attttttgaagcgaaggttgtggatctgga |
| | taccgggaaaacgctgggcgttaatcaaag |
| | aggcgaactgtgtgtgagaggtcctatgat |
| | tatgtccggttatgtaaacaatccggaagc |
| | gaccaacgccttgattgacaaggatggatg |
| | gctacattctggagacatagcttactggga |
| | cgaagacgaacacttcttcatcgttgaccg |
| | cctgaagtctctgattaagtacaaaggcta |
| | tcaggtggctcccgctgaattggaatccat |
| | cttgctccaacaccccaacatcttcgacgc |
| | aggtgtcgcaggtcttcccgacgatgacgc |
| | cggtgaacttcccgccgccgttgttgtttt |
| | ggagcacggaaagacgatgacggaaaaaga |
| | gatcgtggattacgtcgccagtcaagtaac |
| | aaccgcgaaaaagttgcgcggaggagttgt |
| | gtttgtggacgaagtaccgaaaggtcttac |
| | cggaaaactcgacgcaagaaaaatcagaga |
| | gatcctcataaaggccaagaagggcggaaa |
| | gatcgccgtggcggccgcaGCCACTAACTT |
| | CTCCCTGTTGAAACAAGCAGGGGATGTCGA |
| | AGAGAATCCCGGGCCAggtgaaataagaga |
| | gaaaagaagagtaagaagaaatataagagc |
| | caccatgaaatgggtcacatttatatctct |
| | gctcttccttttctcttcagcctacagcca |
| | ggtacagttgcaggagtccggaggtggtct |
| | ggtacaaccaggtggatccctcagattgtc |
| | ttgtgcagctagtggctttacgctcgacta |
| | ctattatatcgggtggtttcggcaagcacc |
| | gggtaaagagagggaggctgttagctgtat |
| | cagcggctcttcagggtccacgtattaccc |
| | tgacagtgttaaagggagatttaccatatc |
| | ccgcgataacgcaaagaacactgtgtactt |
| | gcagatgaatagcctgaagcccgaggacac |
| | agccgtttactactgtgccacgattcgctc |
| | ctcttcatggggaggatgcgttcattacgg |
| | gatggattactggggcaaaggcactcaggt |
| | gacggttagctctggaggcgggggcagcca |
| | cgagaccacccccaacaaggggagcgggac |
| | cacgtccggcacaactagactgctttccgg |
| | ccatacatgctttacacttactgggctgct |
| | ggggactcttgtaactatggggctcctcac |
| | atga |
| Thermal gene | agaagcttcatgaacgtgcagaacgttcat |
| switch driving | gaacgctgagaacgttctagaacctggaga |
| expression of | agcttctagaatgtgctgaaagttctagaa |
| a firefly | cgacgagaacgttctagaaggtctagaacg |
| luciferase and | ttctagaacttgctctagagggtatataa |
| membrane bound | tgggggccactagtctactaccagaaagct |
| 3x Suntag | tggtaccgagctcggatccagccaccctc |
| (TS-Suntag)/ | gagACCGGTatggaagacgccaaaaacata |
| SEQ ID NO: 28 | aagaaaggcccggcgccattctatccgctg |
| | gaagatggaaccgctggagagcaactgcat |
| | aaggctatgaagagatacgccctggttcct |
| | ggaacaattgcttttacagatgcacatatc |
| | gaggtggacatcacttacgctgagtacttc |
| | gaaatgtccgttcggttggcagaagctatg |
| | aaacgatatgggctgaatacaaatcacaga |
| | atcgtcgtatgcagtgaaaactctcttcaa |
| | ttctttatgccggtgttgggcgcgttattt |
| | atcggagttgcagttgcgcccgcgaacgac |
| | atttataatgaacgtgaattgctcaacagt |
| | atgggcatttcgcagcctaccgtggtgttc |
| | gtttccaaaaaggggttgcaaaaaattttg |
| | aacgtgcaaaaaaagctcccaatcatccaa |
| | aaaattattatcatggattctaaaacggat |
| | taccagggatttcagtcgatgtacacgttc |
| | gtcacatctcatctacctcccggttttaat |
| | gaatacgattttgtgccagagtccttcgat |
| | agggacaagacaattgcactgatcatgaac |
| | tcctctggatctactggtctgcctaaaggt |
| | gtcgctctgcctcatagaactgcctgcgtg |
| | agattctcgcatgccagagatcctattttt |
| | ggcaatcaaatcattccggatactgcgatt |
| | ttaagtgttgttccattccatcacggtttt |
| | ggaatgtttactacactcggatatttgata |
| | tgtggatttcgagtcgtcttaatgtataga |
| | tttgaagaagagctgtttctgaggagcctt |
| | caggattacaagattcaaagtgcgctgctg |
| | gtgccaaccctattctccttcttcgccaaa |
| | agcactctgattgacaaatacgatttatct |
| | aatttacacgaaattgcttctggtggcgct |
| | cccctctctaaggaagtcggggaagcggtt |
| | gccaagaggttccatctgccaggtatcagg |
| | caaggatatgggctcactgagactacatca |
| | gctattctgattacacccgagggggatgat |
| | aaaccgggcgcggtcggtaaagttgttcca |
| | ttttttgaagcgaaggttgtggatctggat |
| | accgggaaaacgctgggcgttaatcaaaga |
| | ggcgaactgtgtgtgagaggtcctatgatt |
| | atgtccggttatgtaaacaatccggaagcg |
| | accaacgccttgattgacaaggatggatgg |
| | ctacattctggagacatagcttactgggac |
| | gaagacgaacacttcttcatcgttgaccgc |
| | ctgaagtctctgattaagtacaaaggctat |
| | caggtggctcccgctgaattggaatccatc |
| | ttgctccaacaccccaacatcttcgacgca |
| | ggtgtcgcaggtcttcccgacgatgacgcc |
| | ggtgaacttcccgccgccgttgttgttttg |
| | gagcacggaaagacgatgacggaaaaagag |
| | atcgtggattacgtcgccagtcaagtaaca |
| | accgcgaaaaagttgcgcggaggagttgtg |
| | tttgtggacgaagtaccgaaaggtcttacc |
| | ggaaaactcgacgcaagaaaaatcagagag |
| | atcctcataaaggccaagaagggcggaaag |
| | atcgccgtggcggccgcaGCCACTAACTTC |
| | TCCCTGTTGAAACAAGCAGGGGATGTCGAA |
| | GAGAATCCCGGGCCAgaaataagagagaaa |
| | agaagagtaagaagaaatataagagccacc |
| | atgaaatgggtcacatttatatctctgctc |
| | ttccttttctcttcagcctacagcgaggaa |
| | ctgctgagcaagaactaccacctggaaaac |
| | gaggtggcccggctgaaaaaaggctctggc |
| | tctggcgaagaactgctgtctaagaattat |
| | cacctcgagaatgaggtcgcccgcctcaag |
| | aaaggatctggaagtggcgaggaactcctc |
| | tccaaaaactaccatctcgagaacgaagtc |
| | gctaggcttaagaaaggaggcgggggcagc |
| | cacgagaccacccccaacaaggggagcggg |
| | accacgtccggcacaactagactgctttcc |
| | ggccatacatgctttacacttactgggctg |
| | ctggggactcttgtaactatggggctcctc |
| | acatga |
| Thermal gene | agaagcttcatgaacgtgcagaacgttcat |
| switch driving | gaacgctgagaacgttctagaacctggaga |
| expression of | agcttctagaatgtgctgaaagttctagaa |
| IL-2 (TS-IL2)/ | cgacgagaacgttctagaaggtctagaacg |
| SEQ ID NO: 29 | ttctagaacttgctctagagggtatataa |
| | tgggggccactagtctactaccagaaagct |
| | tggtaccgagctcggatccagccaccctc |
| | gagACCGGTatgtacaggatgcaactcctg |
| | tcttgcattgcactaagtcttgcacttgtc |
| | acaaacagtgcacctacttcaagttctaca |
| | aagaaaacacagctacaactggagcattta |
| | ctgctggatttacagatgattttgaatgga |
| | attaataattacaagaatcccaaactcacc |
| | aggatgctcacatttaagtittacatgccc |
| | aagaaggccacagaactgaaacatcttcag |
| | tgtctagaagaagaactcaaacctctggag |
| | gaagtgctaaatttagctcaaagcaaaaac |
| | tttcacttaagacccagggacttaatcagc |
| | aatatcaacgtaatagttctggaactaaag |
| | ggatctgaaacaacattcatgtgtgaatat |
| | gctgatgagacagcaaccattgtagaattt |
| | ctgaacagatggattaccttttgtcaaagc |
| | atcatctcaacactgacttaa |

Underlined text-7HSE; italic text-YB core promoter; bold text-XhoI restriction site; CAPITALIZED-AgeI restriction site; wavy-underlined text = NotI restriction site; CAPITALIZED UNDERLINED-P2A; bold italic text-gene of interest

In some aspects, the present disclosure provides a vector comprising a promoter construct according to any one of the embodiments disclosed herein. A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment. In some embodiments, the vector is an expression vector. In some embodiments, the vector is a viral vector. Examples of viral vectors include, but are not limited to, adenovirus vectors, adeno-associated virus vectors, retrovirus vectors, gamma retrovirus vectors, and lentivirus vectors. “Retroviruses” are viruses having an RNA genome. “Gamma retrovirus” refers to a genus of the retroviridae family. Examples of gamma retroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

It is understood and herein contemplated that the disclosed promoter constructs are particularly useful in the creation of a adoptive immunotherapy (e.g., T cell) whose therapeutic effects are limited to sites where heat is applied thereby preventing off-site expression and cytotoxicity. Accordingly, disclosed herein are immune cells comprising the promoter construct or vector disclosed herein. In some embodiments, the immune cell is a T cell, natural killer (NK) cell, or dendritic cell. In some embodiments, the T cell is a CD4+ T cell or CD8+ T cell. In some embodiments, the T cell comprises a recombinant TCR. In some embodiments, the immune cell comprises a CAR. In some embodiments, the immune cell is a chimeric antigen receptor (CAR) T cell and/or CAR NK cell. For example, disclosed herein are CAR T cells, CAR NK cells, or recombinant TCR T cells comprising a promoter construct comprising a) one or more heat shock elements (such as, for example, the heat shock element as set forth in SEQ ID NO: 1); b) a core promoter; and c) a gene of interest. In some embodiments, the gene of interest encodes a chimeric antigen receptor, a recombinant TCR, an immunomodulating agent, or any combination thereof.

It is understood and herein contemplated that the disclosed promoter constructs can be applied to a cell to create an immunotherapy against a target. In one aspect, disclosed herein are kits comprising any of the promoter constructs disclosed herein and further comprising a heating element to activate the promoter construct. In some aspects, the heating element can be a light source (such as for example, a laser (including, but not limited to near infrared lasers such as for example, a laser emitting at light between 700 nm to about 1400 nm, such as, for example a laser emitting at 705, 730, 735, 760, 783, 785, 792, 793, 797, 808, 825, 830, 850, 852, 850, 860, 878, 880, 885, 888, 891, 900, 905, 915, 938, 940, 946, 960, 975, 976, 980, 1030, 1040, 1053, 1064, 1123, 1177, 1210, 1280, 1300, 1317, 1319, and/or 1370 nm), filament, infrared emitting light source, or light emitting diode (LED)), thermal pad, or thermally regulated needle, probe, or scalpel.

### Methods of Treating Cancer

The disclosed promoter constructs, vectors, and immune cells (e.g., recombinant TCR T cells, CAR T cells, and/or CAR NK cells) comprising said promoter constructs can be used to treat any disease where uncontrolled cellular proliferation occurs such as cancers. A representative but non-limiting list of cancers that the disclosed compositions can be used to treat is the following: sarcomas, blastomas, lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

Accordingly, disclosed herein are methods of treating, reducing, decreasing, inhibiting, ameliorating, and/or preventing a cancer and/or metastasis (such as for example, a solid tumor including, but not limited to, epithelial carcinoma, a sarcoma, a lymphoma, a blastoma, or a melanoma) in a subject comprising administering to the subject any of the promoter constructs, vectors, immune cells (e.g., recombinant TCR T cells, CAR T cells, or CAR NK cells), and/or utilizing any of the kits disclosed herein. For example, disclosed herein are methods of treating, reducing, decreasing, inhibiting, ameliorating, and/or preventing a cancer and/or metastasis (such as for example, a solid tumor including, but not limited to, epithelial carcinoma, a sarcoma, a lymphoma, a blastoma, or a melanoma) in a subject comprising: i) administering to the subject a thermally controlled immune cell (such as, for example, a recombinant TCR T cell, a CAR T cell or CAR NK cell) comprising a promoter construct comprising a) one or more heat shock elements (such as, for example, the heat shock element as set forth in SEQ ID NO: 1); b) a core promoter (such as, for example, a core promoter comprising a heat shock protein transcription start site encoding HSPA1A, HSPH1, HSPB1, HSP6, HSP70, HSPA6, or YB); and c) a gene of interest (such as, for example, a reporter gene, an immunomodulating agent, a bispecific T cell engager (BITE) (such as, for example, a bispecific T cell engager antibody comprising an anti-CD-3 binding domain and an NKG2D receptor extracellular domain), a recombinant TCR, a chimeric antigen receptor (CAR), or any combination thereof) and ii) inducing activation of the immune cell at the site of the tumor (such as, for example, heating the immune cell to between 40° C.-45° C. (such as, for example, between 40° C. and 42° C. or between 41° C. and 43° C. or between 42° C. and 45° C., including, but not limited to 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some embodiments the promoter requires a thermal activation of at least 40.0, 40.1, 40.2, 40.3, 40.4, 40.5, 40.6, 40.7, 40.8, 40.9, 41.0, 41.1, 41.2, 41.3, 41.4, 41.5, 41.6, 41.7, 41.8, 41.9, 42.0, 42.1, 42.2, 42.3, 42.4, 42.5, 42.6, 42.7, 42.8, 42.9, 43.0, 43.1, 43.2, 43.3, 43.4, 43.5, 43.6, 43.7, 43.8, 43.9, 44.0, 44.1, 44.2, 44.3, 44.4, 44.5, 44.6, 44.7, 44.8, 44.9, or 45.0° C.). In some aspects, the heating can be achieved by applying a heating element to activate the promoter construct. In some aspects, the heating element can be a light source (such as for example, a laser (including, but not limited to near infrared lasers such as for example, a laser emitting at light between 700 nm to about 1400 nm, such as, for example a laser emitting at 705, 730, 735, 760, 783, 785, 792, 793, 797, 808, 825, 830, 850, 852, 850, 860, 878, 880, 885, 888, 891, 900, 905, 915, 938, 940, 946, 960, 975, 976, 980, 1030, 1040, 1053, 1064, 1123, 1177, 1210, 1280, 1300, 1317, 1319, and/or 1370 nm), filament, infrared emitting light source, or light emitting diode (LED)), thermal pad, or thermally regulated needle, probe, or scalpel.

In some aspects, the method can further comprise administering an additional anticancer agent or immunotherapy (including, but not limited checkpoint inhibitor such as anti-PD-1 immunotherapy, anti-PD-L1 immunotherapy, anti-CTLA-4 immunotherapy). Immunotherapy targeting an immune checkpoint molecule may be an antibody or antigen binding fragment thereof, or an antibody fusion protein.

It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BICNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF. Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin. Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V. Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHlg.M12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT)(such as, for example BMS-986207, OMP-313M32, MK-7684, AB-154, ASP-8374, MTIG7192A, or PVSRIPO), CD96,—and T-lymphocyte attenuator (BTLA), V-domain Ig suppressor of T cell activation (VISTA)(such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1

### Results

### Engineering Thermal-Specific Gene Switches

The cellular response to hyperthermia is mediated by trimerization of the temperature-sensitive transcription factor Heat Shock Factor 1 (HSF1) and its subsequent binding to HSEs. HSEs comprise multiple inverted repeats of the consensus sequence 5′-nGAAn-3′ and are arrayed upstream of the transcription start site of heat shock proteins (HSPs) to enable their upregulation following thermal stress. The response of endogenous HSP genes is selective, but not specific, for heat as their promoters contain additional regulatory elements (e.g., hypoxia response elements, metal-responsive elements) that mediate transcription following exposure to a diverse set of cues including hypoxia, heavy metals, and mechanical force. Moreover, differences in the core promoter (e.g., initiator elements, TATA box) influence the composition of the pre-initiation complex (PIC) and its interactions with transcriptional enhancers including HSF1, leading to different thermal responses across tissues and types of cells. Due to this complexity and cross-activation by non-thermal response pathways, synthetic gene switches are activated by heat but not by other sources of stress.

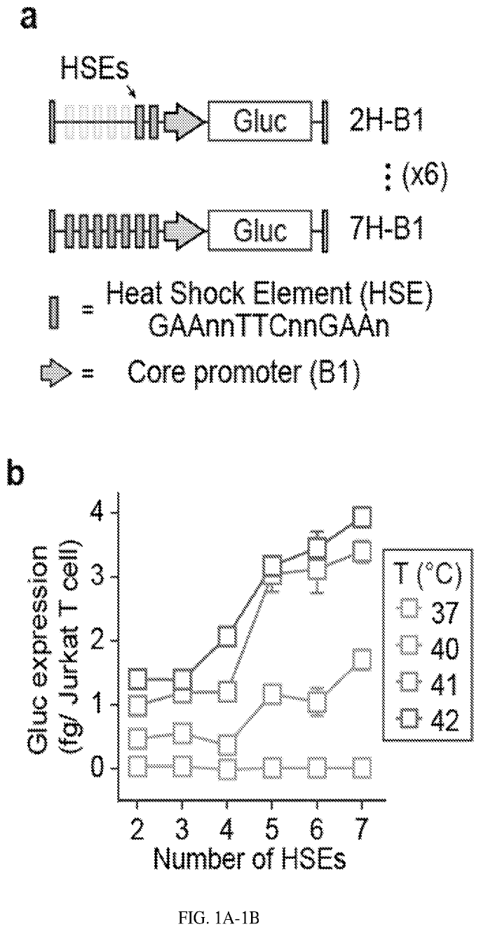

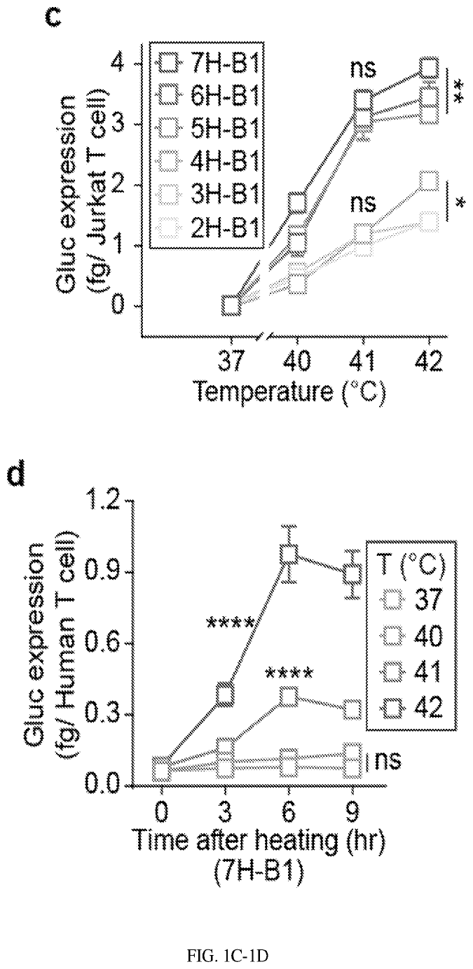

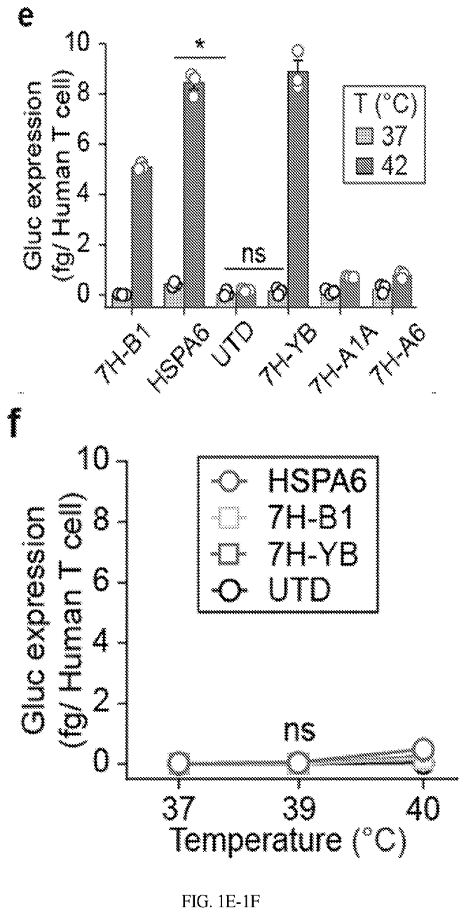

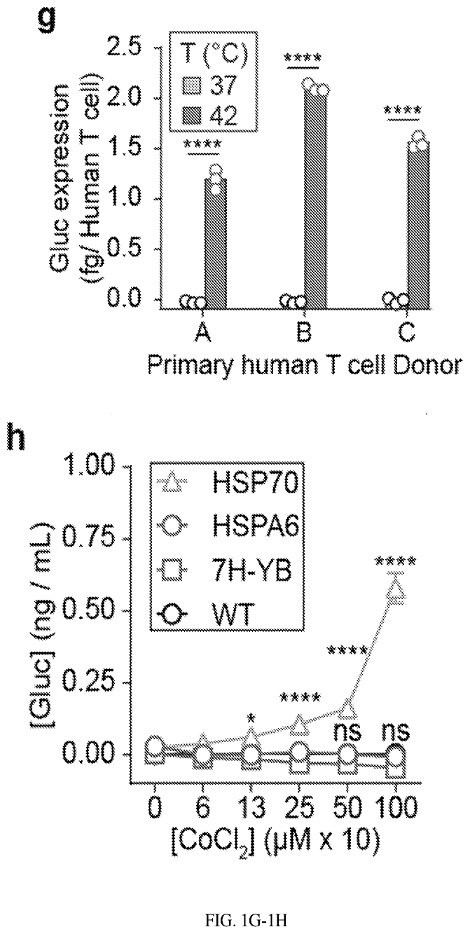

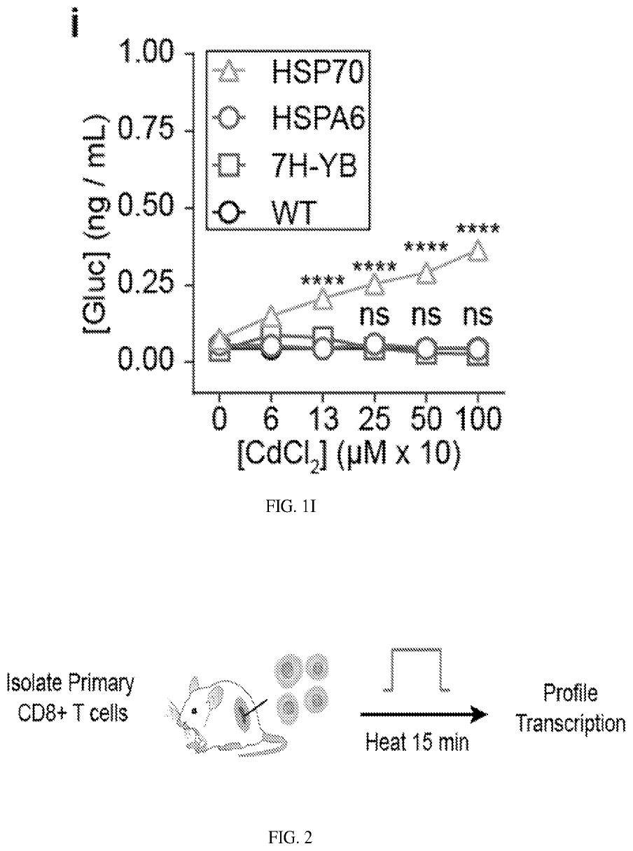

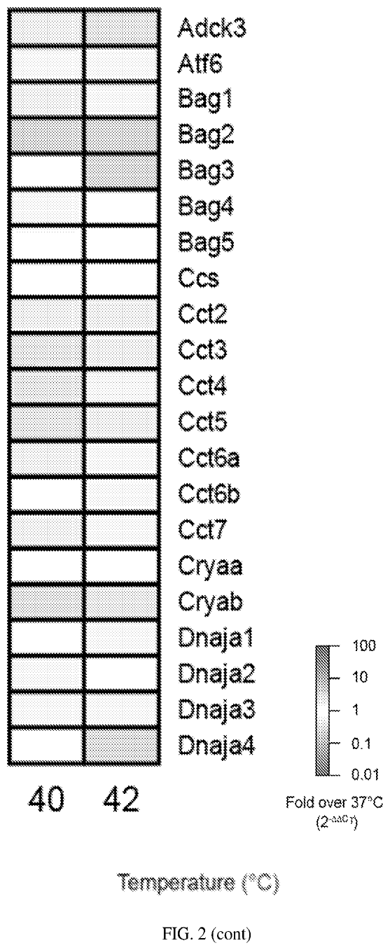

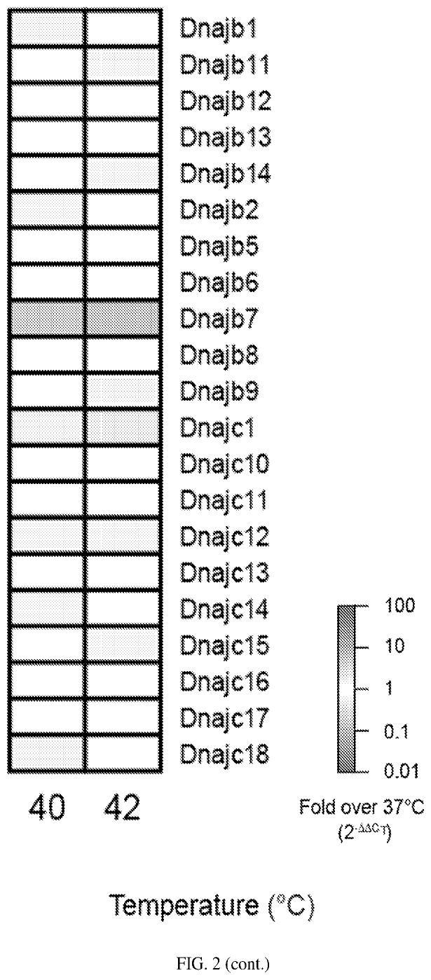

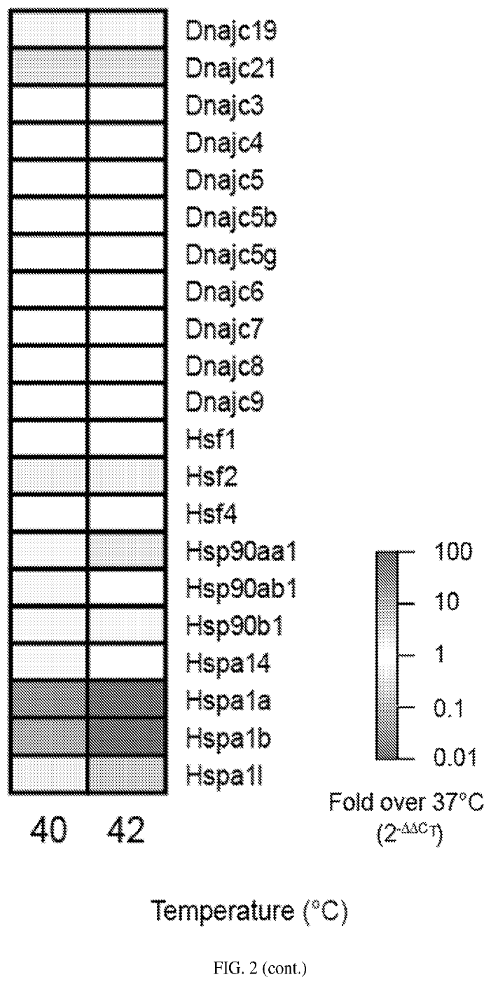

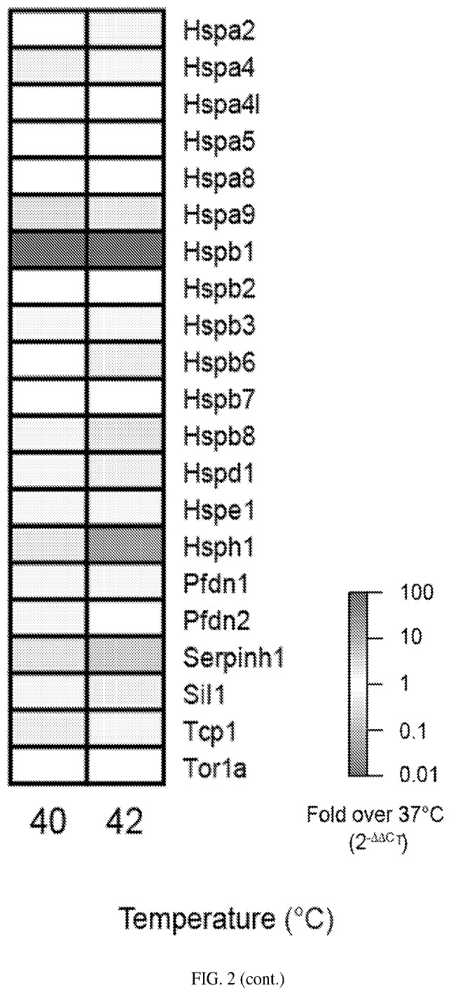

Six candidate constructs were cloned comprising 2 to 7 repeats of the HSE motif 5′-nGAAnnTTCnnGAAn-3′ upstream of the HSPB1 core promoter into Jurkat T cells (labeled 2H-B1 to 7H-B1, FIG. 1a). The HSPB1 core promoter was initially selected as its parent gene was one of two that were upregulated by more than 20-fold at 42° C. in primary murine T cells in contrast to more than 80 HSP and HSP-related genes that did not respond to heat treatment (FIG. 2). Selecting a core promoter from an endogenous gene with high thermal response facilitates transcriptional activity when integrated with HSE repeats. To quantify responses of the thermal switches, transduced Jurkat T cells were transiently heated to 3-5° C. above body temperature (i.e., 40-42° C.), which is a mild temperature range in contrast to those used for ablative therapies (>50° C.)²⁵. Compared to control samples at 37° C., increased expression of the reporter Gaussia luciferase (Gluc) was observed as the temperature and number of HSEs increased (FIG. 1b). Constructs containing 5-7 HSE repeats (5H-B1 to 7H-B1) resulted in significantly higher thermal responses compared to those with 2-4 HSEs (2H-B1 to 4H-B1) (FIG. 1c).

To test thermal response in primary human T cells, T cells were transduced with the 7H-B1 construct and observed peak thermal activity approximately 6 hours after heating at temperatures above 40° C. (FIG. 1d). Because the HSPB1 core promoter was initially selected from a screen of murine T cells, the thermal responses in primary human T cells further depend on the core promoter sequence. Thus, core promoters identified and compared in the qPCR screen (A1A, A6, B1) (FIG. 2), from the human HSPA6 gene based on past work, and a synthetic core promoter (YB). Among the panel, the 7H-YB construct resulted in the highest increase in Gluc reporter levels after 30 minutes at 42° C., corresponding to a ˜60-fold increase in activity (FIG. 1e). Basal activity at 37° C. remained statistically identical to untransduced controls, and negligible activation was observed at temperatures 37-40° C. that correspond to fever range for 24 hours (FIG. 1f). 7H-YB thermal activation was further verified in T cells derived from three separate donors to confirm lack of donor-dependency (FIG. 1g; FIG. 3). Based on this data, 7H-YB was selected for subsequent experiments.

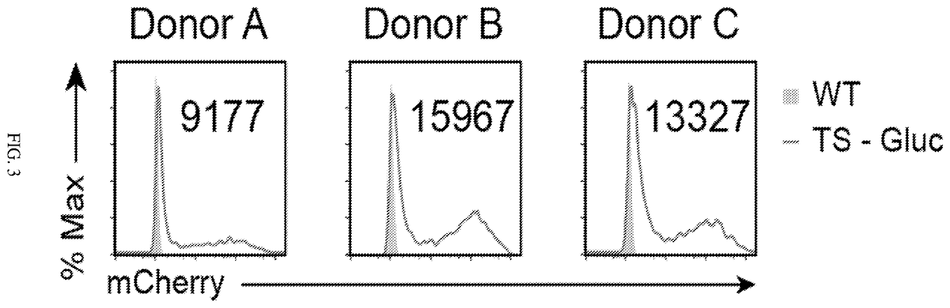

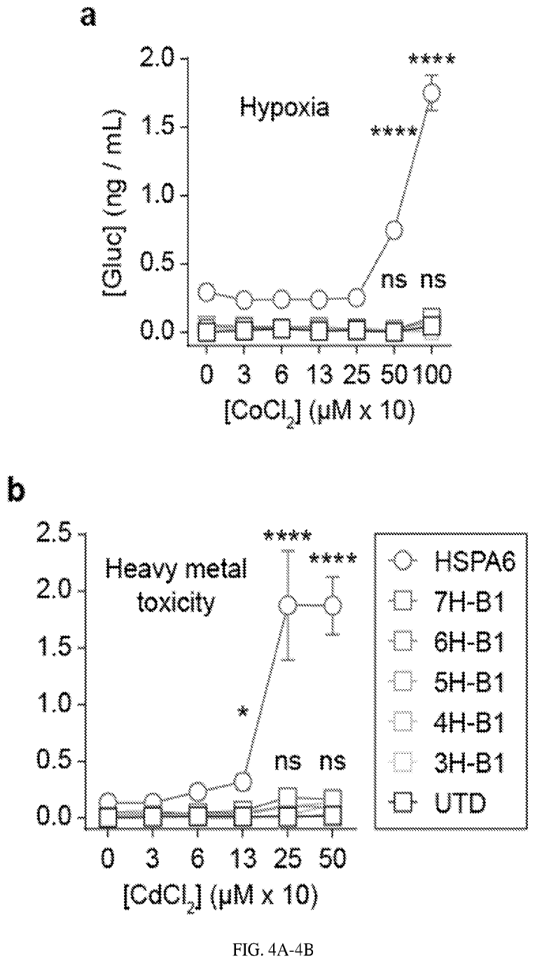

We tested thermal specificity using hypoxia and heavy metal toxicity as two representative non-thermal stresses. As a benchmark, endogenous HSPA6 or HSP70 promoters were compared, which are highly stress-inducible and previously used for thermal control of gene expression. The gene switches were tested by incubating transduced primary human T cells and Jurkat T cells with the hypoxia-mimetic agent CoCl₂, a stabilizer of the hypoxia response's master regulator Hypoxia Inducible Factor-1α (HIF-1α), as well as the heavy metal complex cadmium chloride (CdCl₂), which accumulates in the body by diet or environmental exposure. While the HSP70 or HSPA6 promoter showed dose-dependent activation by hypoxia and cadmium toxicity in primary human T cells or Jurkat T cells respectively (FIG. 1h, i; FIG. 4), 7H-YB was not activated and remained statistically identical to untransduced (UTD) controls up to concentrations above the ranges commonly used to test cellular responses to hypoxia and cadmium (1000 mM CoCl₂ and 1000 μM CdCl₂). These results show that these constructs have increased thermal-specificity when exposed to non-thermal stresses compared to endogenous HSPs.

Primary T Cells Maintain Key Functions after Thermal Treatments

Next, thermal delivery profiles were identified that would be well-tolerated by primary T cells without affecting key functions including proliferation, migration, and cytotoxicity. In thermal medicine, heating target sites to temperatures greater than 50° C. is used to locally ablate tissue by inducing tumor cell apoptosis and coagulative necrosis. By contrast, mild hyperthermia therapy (40-42° C.) is used to enhance transport of small molecules such as in Hyperthermic Intraperitoneal Chemotherapy (HIPEC) where abdominal infusions of heated chemotherapy serve as adjuvant treatment following surgical debulking in advanced ovarian cancer patients. At temperatures below 45° C., transient exposure to mild hyperthermia is well-tolerated by cells and tissues due to induction of stress-response pathways including HSPs. In addition, T cell responses to continuous and fractionated heat treatments were considered. Dose fractionation is a commonly used in radiotherapy to reduce damage to normal tissues while maximizing the effect of radiation on cancer. Based on previous observations that thermal pulse trains increased Jurkat T cell tolerance compared to continuous heat treatments with an identical treatment area-under-the-curve (AUC), the effect of thermal dose fractionation on primary T cells was further investigated.

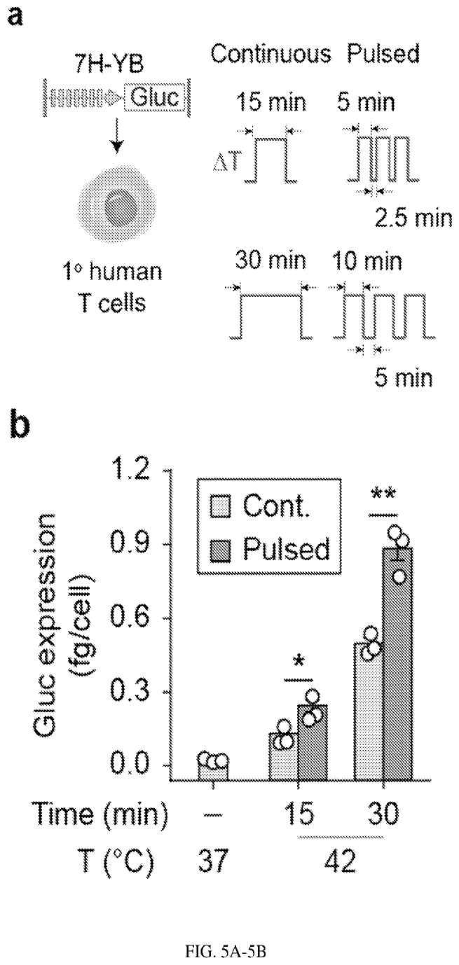

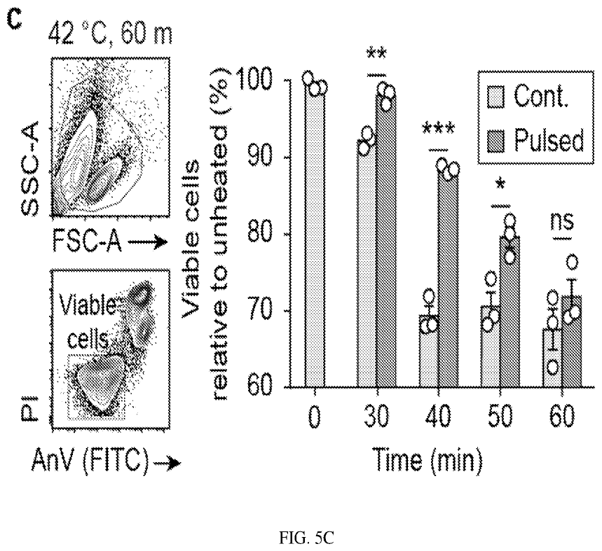

Pulsed heat treatments at 67% duty cycles comprising of three discrete thermal pulses (5 or 10 min each) separated by intervening rest periods at 37° C. (2.5 or 5 min each) were compared to their unfractionated counterparts (15 or 30 min continuous heating) (FIG. 5a). In primary human T cells transduced with the 7H-YB Gluc vector, pulsed heat treatments resulted in significantly higher reporter expression by up to ˜87% compared to continuous delivery at a 30 minute AUC (FIG. 5b). To assess T cell viability, cell death (PI) and apoptotic (Annexin V) markers was quantified and significant improvements were observed for primary T cells that received pulsed treatments at a 67% duty cycle for durations from 30 to 60 minutes (FIG. 5c). By contrast, as high as ˜33% reduction in T cell viability was observed in samples that received continuous heat treatments for greater than 40 minutes. Similar trends were observed in T cell proliferation assays by dye (CTV) dilution where the percent of proliferated T cells following incubation with CD3/28 beads was unaffected by both continuous and pulsed heating for 30 minutes at 42 or 43° C. while samples that were heated for 60 minutes resulted in reduced T cell proliferation (FIG. 5d; FIG. 6).

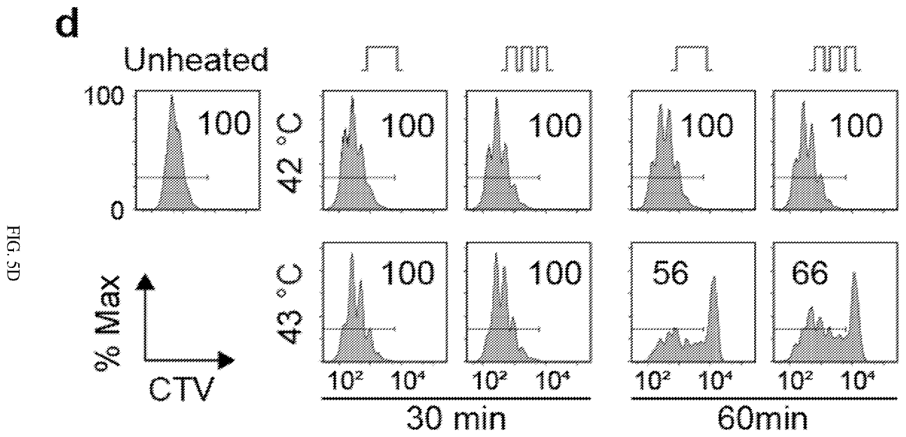

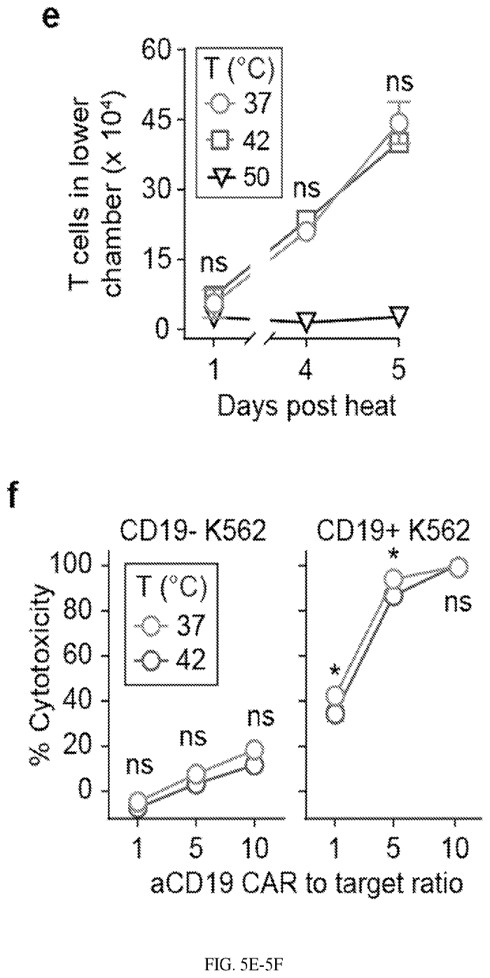

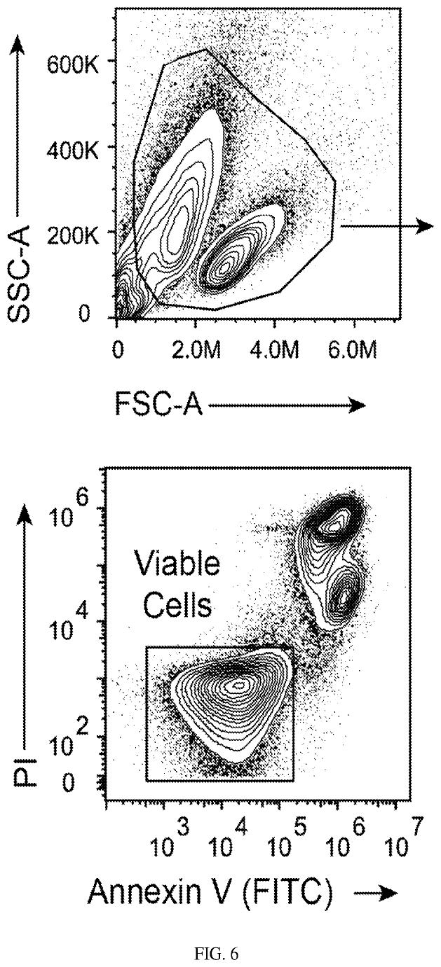

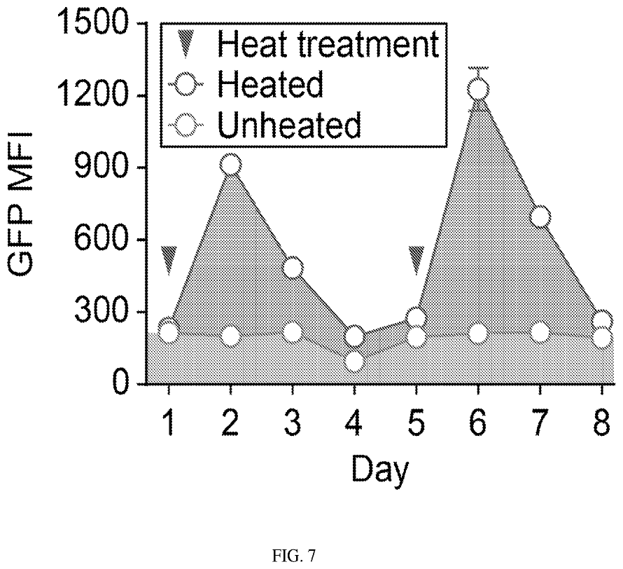

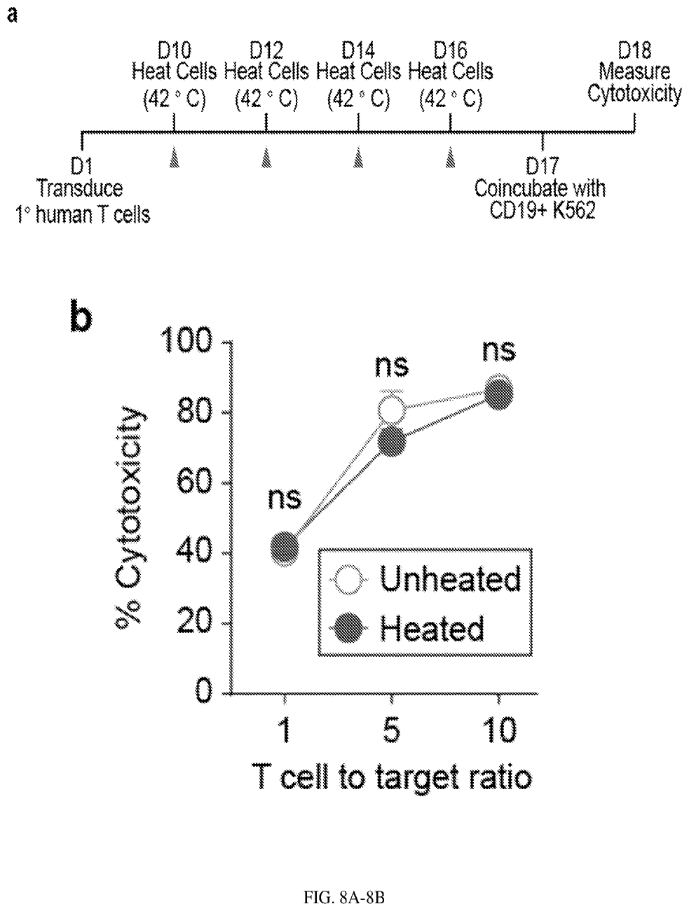

To probe T cell migration by chemotaxis, transwell assays were used and heat treatments were observed that (42° C. for 30 min) did not significantly affect T cell migration into lower wells containing the chemokine CXCL12 whereas T cells heated to 50° C. were affected (FIG. 5e). To test longitudinal activation, T cells were re-heated over the course of 8 days and observed similar increases in GFP mean fluorescent intensity (MFI), as well as GFP activation and decay half-lives (t_{1/2}˜0.5 and 1 day, respectively), indicating that the magnitude and kinetics of T cell responses are unaffected by multiple heat treatments (FIG. 7). To quantify the effect of heat on T cell cytotoxicity, primary human T cells expressing an αCD19 CAR under a constitutive EF1α promoter were incubated with either CD19+ or CD19-K562s containing a luciferase reporter to allow quantification of cell death by loss of luminescence (FIG. 5e). At all effector to target cell ratios tested (1:1, 5:1, 10:1), heated T cells maintained greater than 90% of the cytotoxicity observed in unheated samples while no significant difference in cytotoxicity was observed in samples containing CD19-K562 target cells (FIG. 5f). Similarly, no statistical difference was observed with longitudinal heat treatments where αCD19 CAR T cells were heated 4 times over the course of 8 days prior to coincubation with CD19+ K562 cells (FIG. 8). Collectively, these data demonstrate that primary human T cells maintain the ability to proliferate, migrate, and kill target cells following short heat treatments delivered in continuous or pulsed wave forms for less than 30 minutes in duration.

### Photothermal Activation of T Cells In Vivo

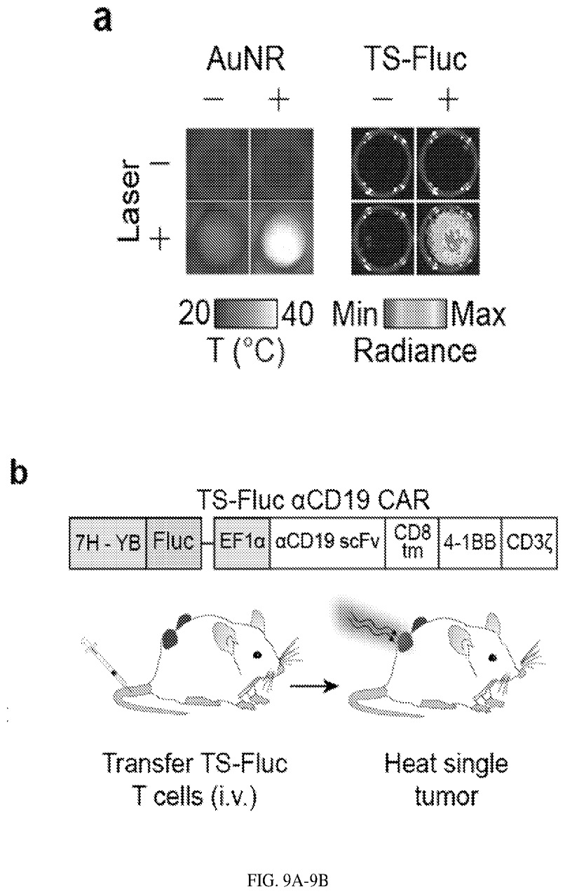

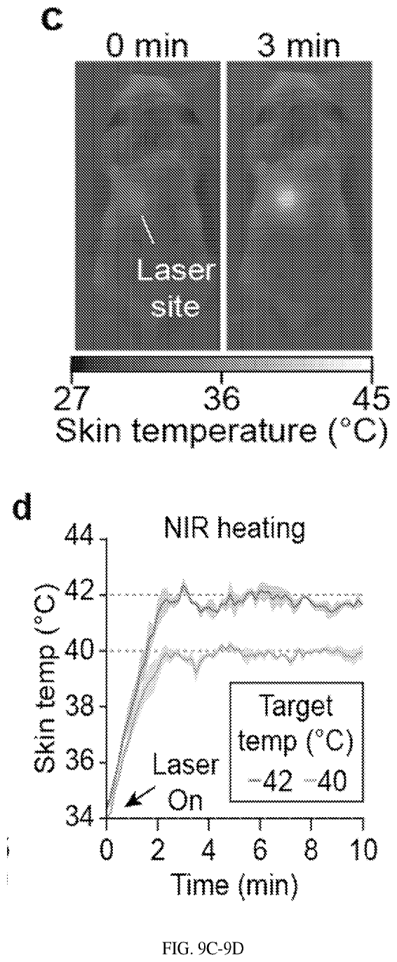

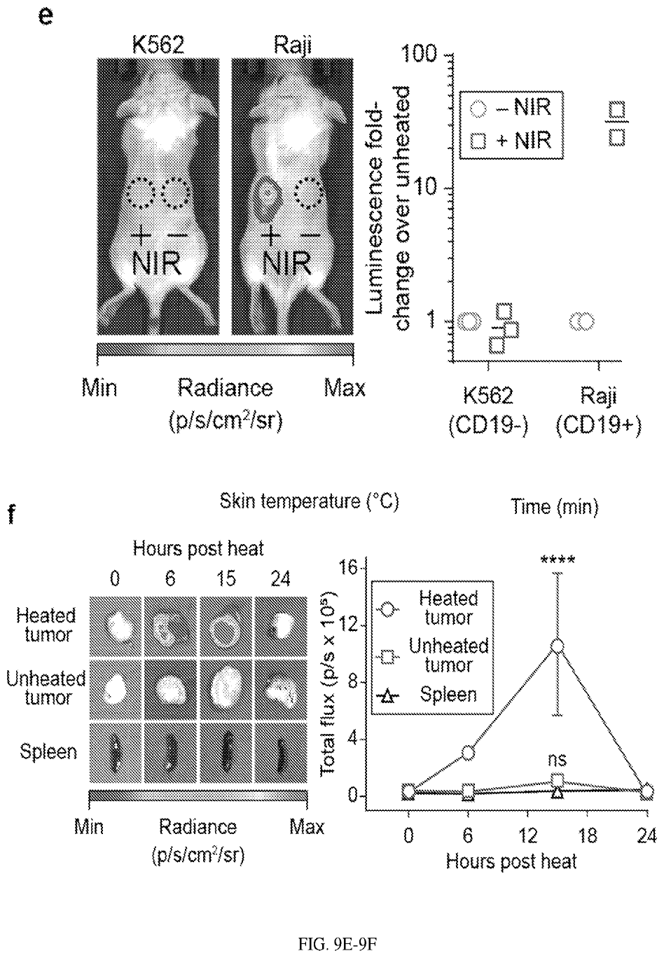

We next sought to demonstrate spatially targeted activation of adoptively transferred T cells by photothermal heating. To locally heat tumors, plasmonic gold nanorods (AuNRs) were used as antennas to convert incident near infra-red (NIR) light (˜650-900 nm) into heat. PEG-coated AuNRs are well-studied nanomaterials with long circulation times that passively accumulate in tumors following intravenous administration. To confirm photothermal heating and thermal switch activation, primary T cells transduced with 7H-YB Fluc (TS-Fluc) αCD19 CAR were co-incubated with AuNRs in 96-well plates and irradiated with 808 nm laser light. In wells that reached 40-45° C. as monitored by a thermal camera, a marked increase in luminescent signals was observed after 6 hours when TS-Fluc αCD19 CAR T cells were present but not in wells containing untransduced controls (FIG. 9a), confirming plasmonic photothermal control of engineered T cells.

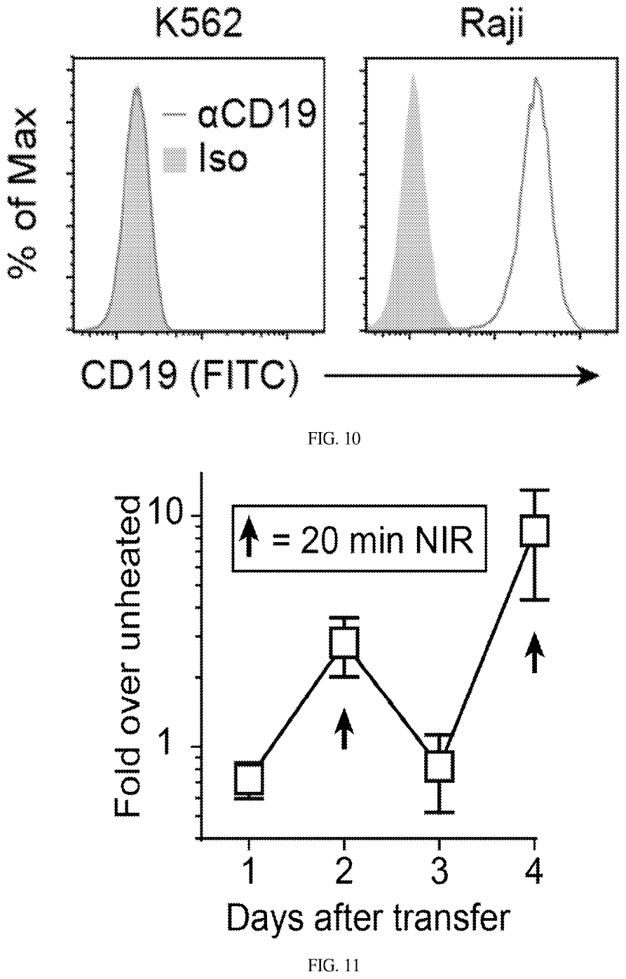

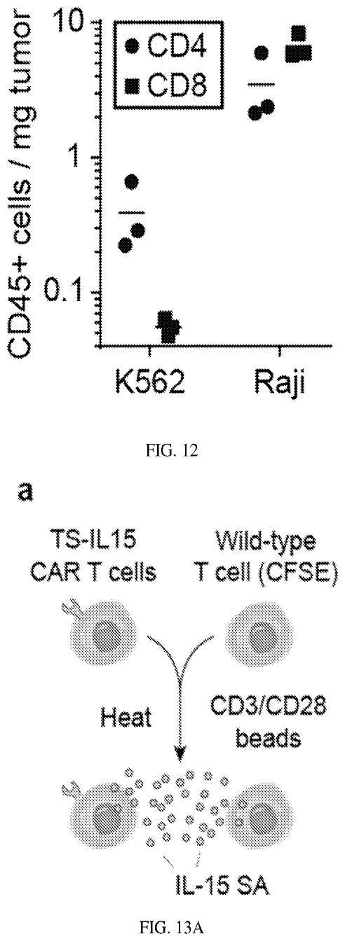

To implement photothermal targeting in vivo, NSG mice with bilateral flank tumors were inoculated with one cohort receiving CD19-K562 cells and a separate cohort receiving CD19+ Raji cells to model CAR antigen negative and positive tumors respectively (FIG. 10). Following intravenous injection of AuNRs and adoptive transfer of TS-Fluc αCD19 CAR T cells (FIG. 9b), tumors were irradiated with NIR laser light under the guidance of a thermal camera (FIG. 9c) to maintain target skin temperatures (FIG. 9d). After 20-minute heat treatments, luminescence increased by more than 30-fold in Raji tumors that received NIR light compared to unheated tumors in the same animal (FIG. 9e) and similar to in vitro experiments, thermal activation repeated twice over the course of 4 days did not result in loss of luminescent signals (FIG. 11). By contrast, increased luminescence was not observed in K562 tumors that were treated with or without NIR light (FIG. 9e). This lack of heat-induced activity was attributed to the absence of CD19 CAR antigen, which resulted in a 20-fold lower density of intratumoral αCD19 CAR T cells in resected K562 tumors compared to CD19+ Raji tumors (FIG. 12). Although heat-triggered expression of transgenes is spatially controlled by photothermal targeting, heat activated T cells migrate out of tumors and therefore result in off-target expression of transgenes. Therefore, in mice bearing bilateral CD19+ Raji flank tumors, a single tumor site was heated and Fluc activity quantified in the distal tumor and the spleen. Whereas luminescence in heated tumors increased by approximately 40-fold within 15 hours after heating, unheated tumors and spleens were statistically identical to baseline levels, indicating that transgene expression in TS-Fluc αCD19 CAR T cells was spatially confined to the site that was heated (FIG. 9f). Collectively these data demonstrated photothermal control of intratumoral T cells engineered with thermal gene switches.

### Remote Thermal Control of IL-15 SA Enhances Adoptive T Cell Transfer

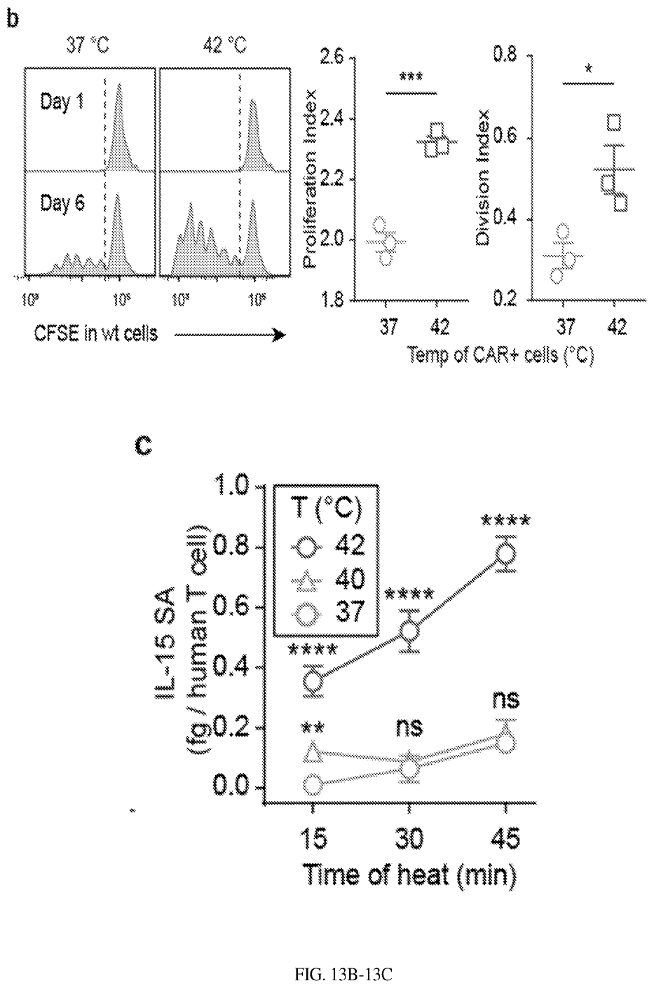

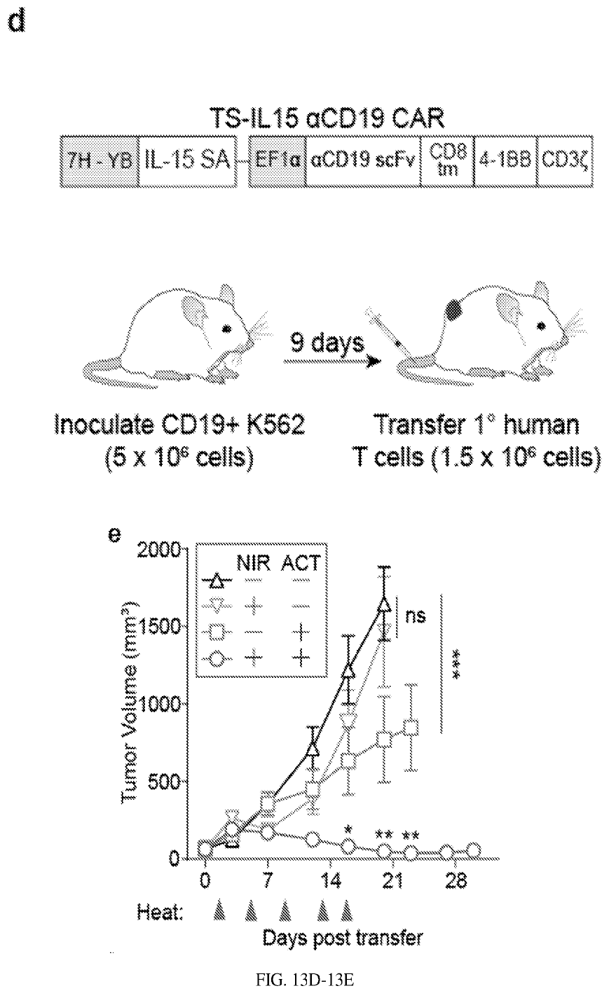

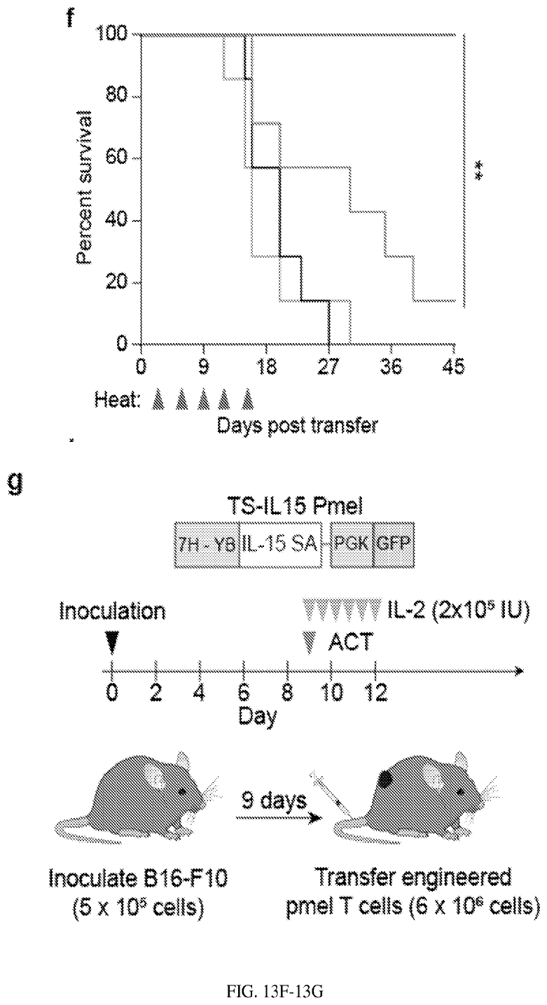

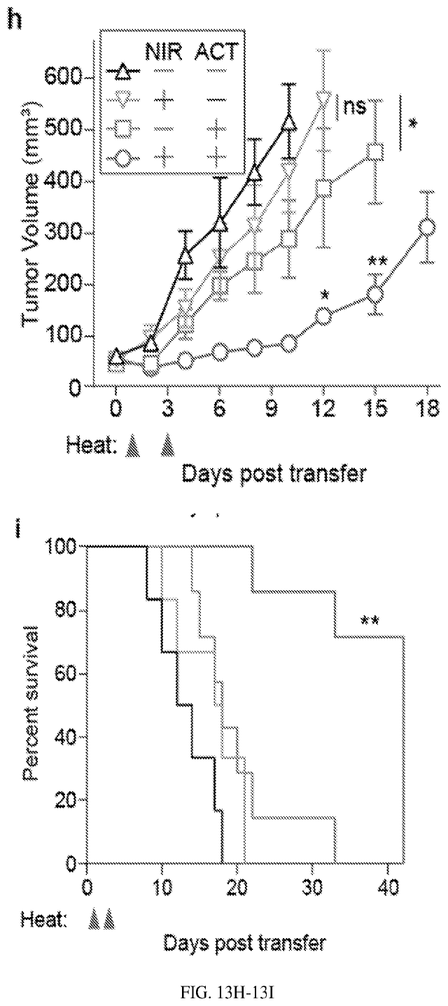

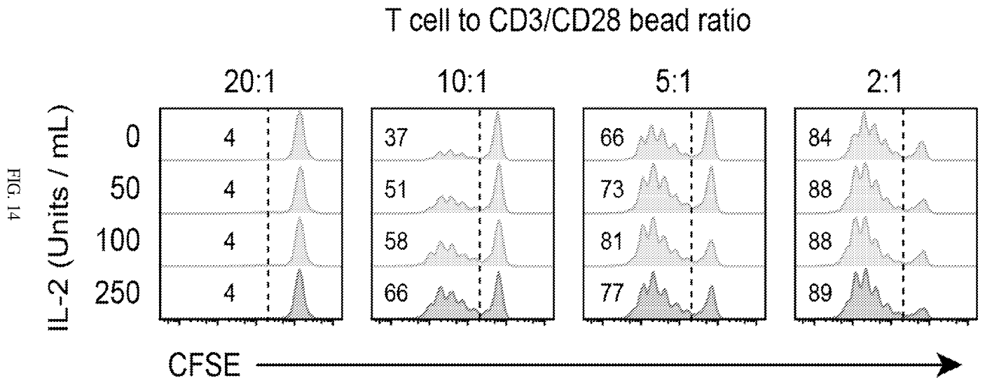

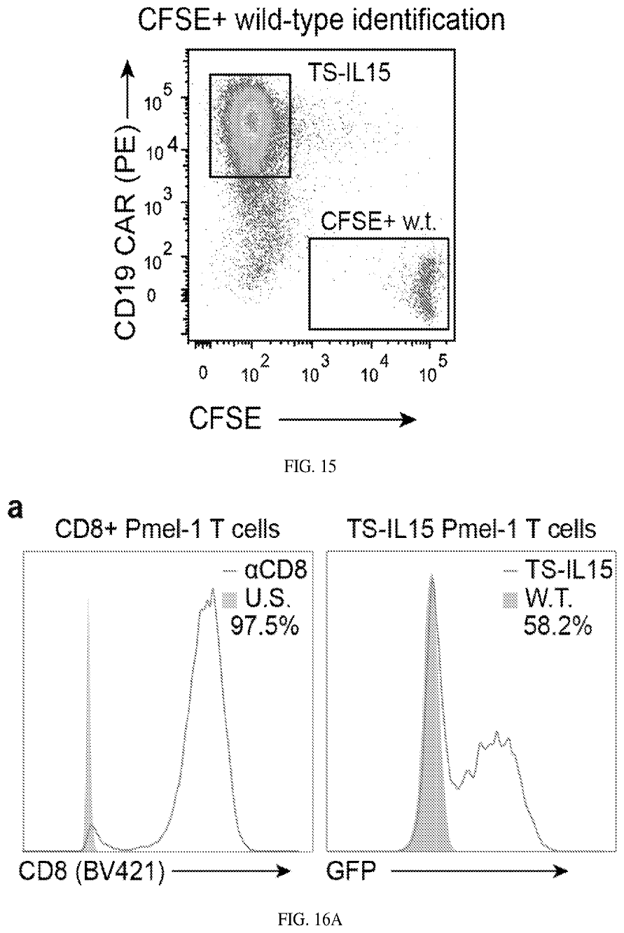

Next, it was investigated whether thermal control enhances the effectiveness of adoptive T cell therapies in vivo. To do this, a single-chain IL-15 superagonist (IL-15 SA) was cloned comprising of the cytokine tethered to the sushi domain of the IL-15Rα subunit under control of our thermal vector (TS-IL15). IL-15 SA is a potent stimulant of CD8 T cells and NK cells and a clinical candidate, ALT-803, is currently under investigation for a wide range of cancers. To test whether heat-induced IL-15 SA was functionally active, a T cell proliferation assay was developed using CFSE-labeled wild-type T cells incubated with CD3/28 beads at a 10:1 ratio without supplemental cytokines. This condition was found to be insufficient to induce T cell proliferation compared to conditions when cytokines such as IL-2 was present in media (FIG. 14). Therefore, to test thermal control of IL-15 SA, heated or unheated TS-IL15 αCD19 CAR T cells were added to samples containing CFSE-labeled wild-type T cells with CD3/28 beads at a 10:1 T cell to bead ratio (FIG. 13a; FIG. 15). Compared to unheated controls, CFSE-labeled T cells in heated samples were found to expand with significantly higher proliferation and division indices (FIG. 13b), demonstrating that TS-IL15 αCD19 T cells can produce physiologically active levels of IL-15 SA following a single thermal treatment. To further characterize the thermal effect of heat-triggered secretion of IL-15 SA, conditioned media was analyzed by ELISA and found that IL-15 SA levels increased with the duration and temperature of thermal treatment (FIG. 13c).

To explore the therapeutic effect by thermal targeting, TS-IL15 αCD19 CAR T cells were adoptively transferred into NSG mice bearing CD19+ K562 tumors when tumors averaged 70 mm³ in volume (FIG. 13d). Photothermal heating of tumors was then carried out every 3-4 days after ACT (days 2, 6, 9, 13, and 16) for a total of five treatments. Compared to control mice that did not receive CAR T cells or heat treatments (black), thermal treatment of tumor sites alone did not lead to reduction in tumor burden or improvement in survival (gray) (FIG. 13e-f). Transfer of TS-IL15 αCD19 CAR T cells alone significantly reduced tumor burden (blue) yet greater than 85% (6/7) of animals reached euthanasia criteria within 39 days of ACT. By contrast, ACT of TS-IL15 αCD19 CAR T cells combined with NIR treatments markedly reduced tumor burden and no animals reached euthanasia criteria within the time window of the study.

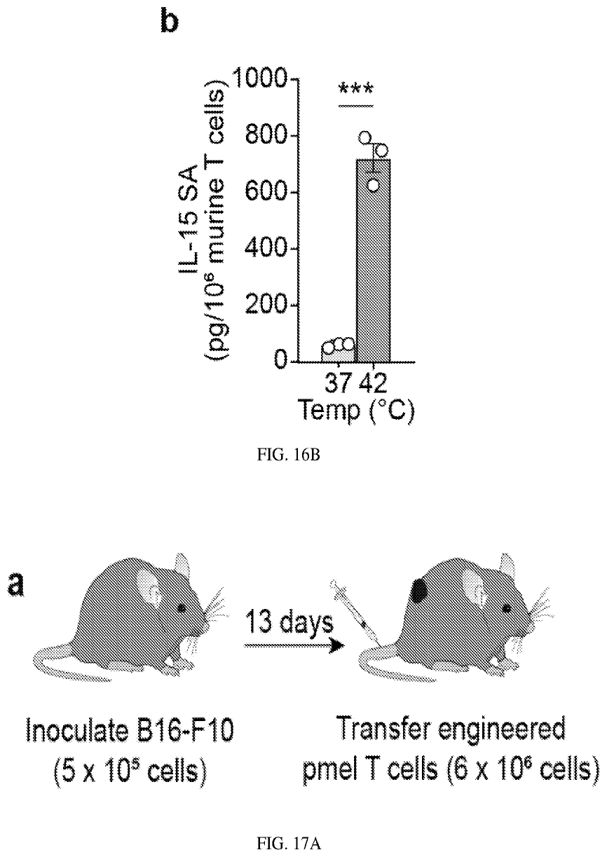

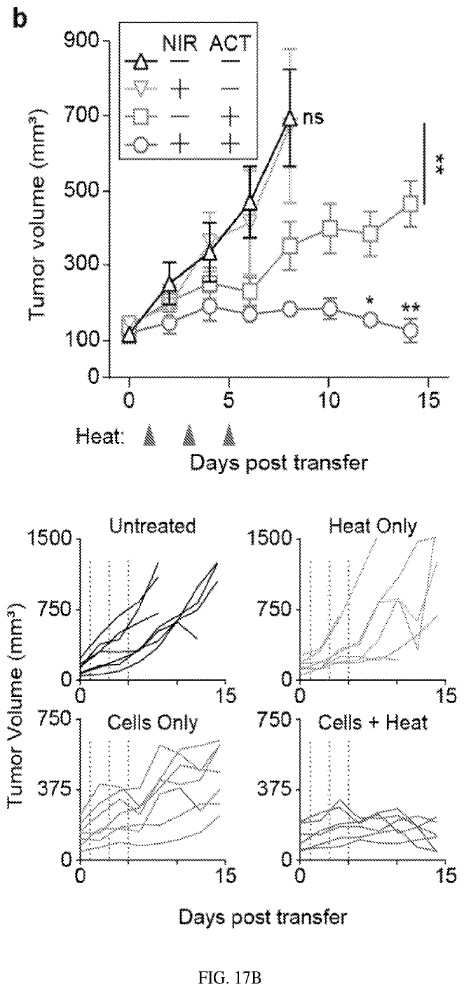

As NSG mice lack an intact immune system, this platform was further tested in immunocompetent C57BL/6J mice bearing syngeneic B16-F10 melanoma tumors with transgenic TCR Pmel-1 T cells that recognize the melanoma self-antigen gp100 (FIG. 13g). Following peptide activation of Pmel-1 splenocytes with hGP100₂₅₋₃₃, CD8+ purity, transduction efficiency, and thermal production of IL-15 SA were verified (FIG. 16). TS-IL15 Pmel-1 T cells were adoptively transferred on day 9 after lymphodepletion (˜52 mm³ average tumor volume) and 2×10⁵ IU of IL-2 was given twice a day for three days to expand transferred cells. Under these conditions, two cycles of photothermal treatment were observed (day 1 and 3 post ACT) to lead to significantly enhanced control of tumor growth (red) compared to cohorts that received TS-IL15 Pmel-1+IL-2 but without heat treatment (blue), heat treatments only (grey), or untreated animals (FIG. 13h). Whereas all control mice reached euthanasia criteria within 33 days after ACT, photothermal treatment of TS-IL15 Pmel-1 T cells resulted in significantly extended survival to day 42 (FIG. 13i). The experiments were repeated with well-established and vascularized B16-F10 tumors (˜120 mm³ average tumor volume) and likewise observed significant improvements in tumor control in heat-treated mice (FIG. 17). These results are consistent with previous studies that showed IL-2 and IL-15 in combination improves antitumor activity compared to treatment with IL-2 alone. Together, these data indicated that photothermal control of IL-15 SA production by CAR or TCR engineered T cells significantly improves tumor control.

### TS-BiTE αHER2 CAR T Cells Mitigate Antigen Escape

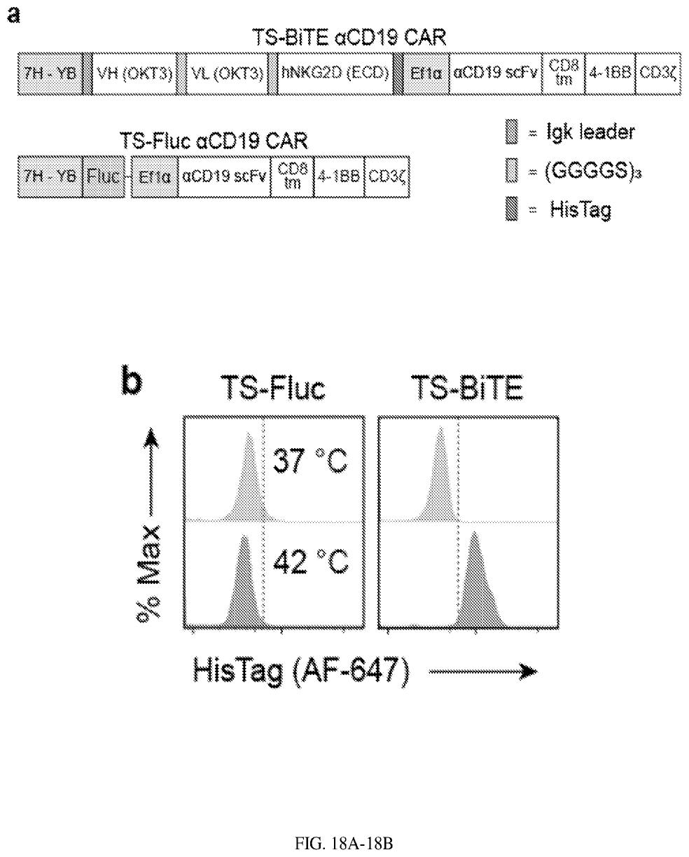

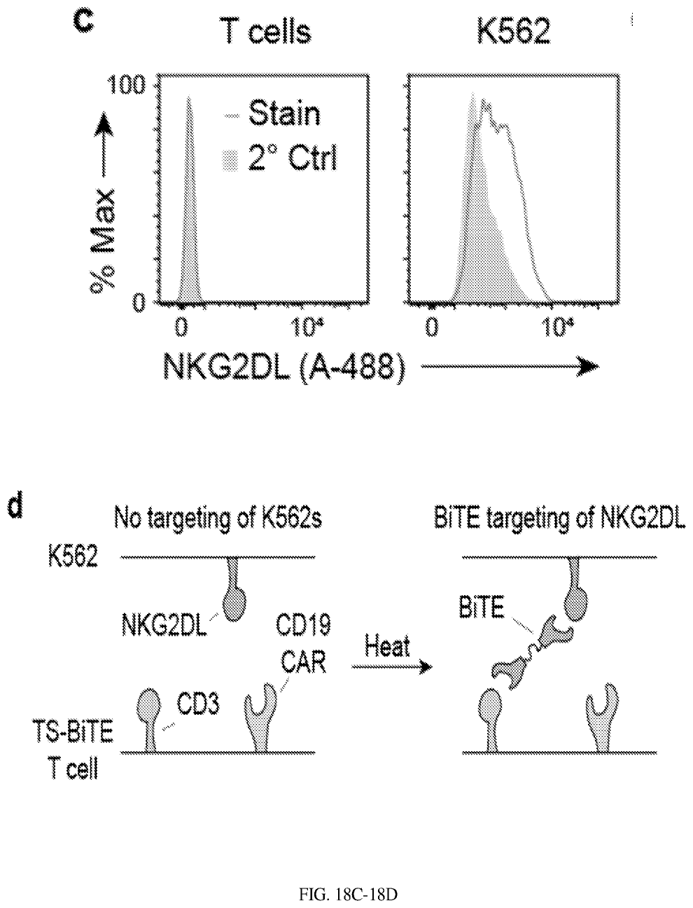

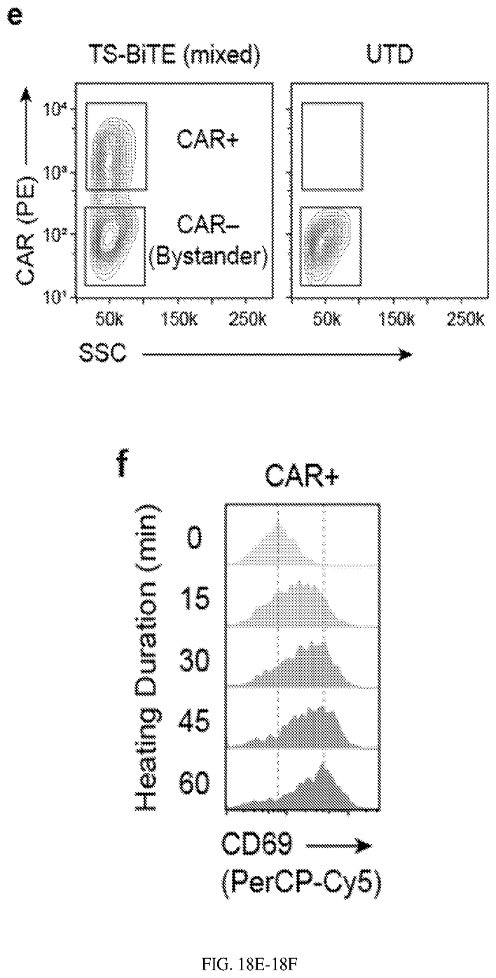

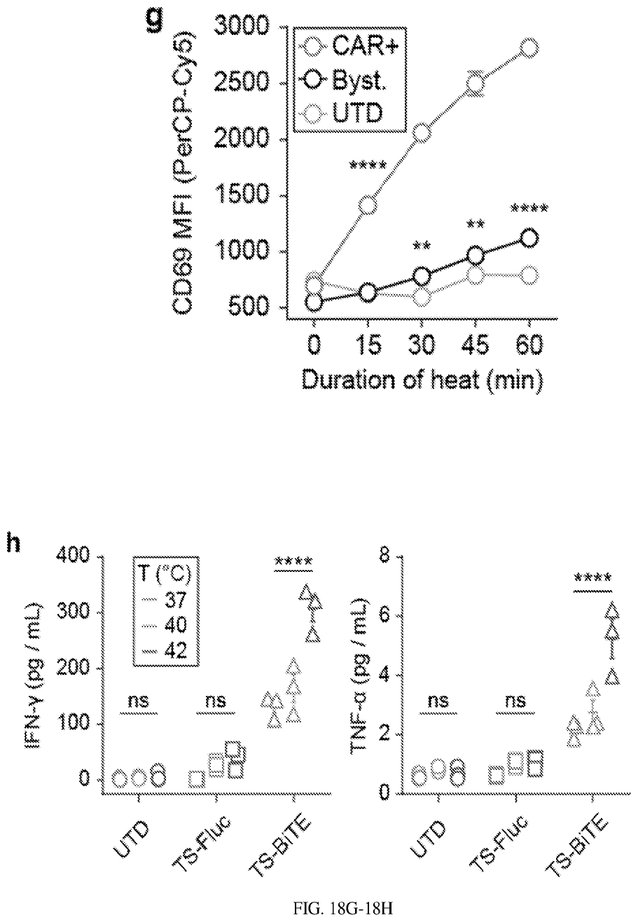

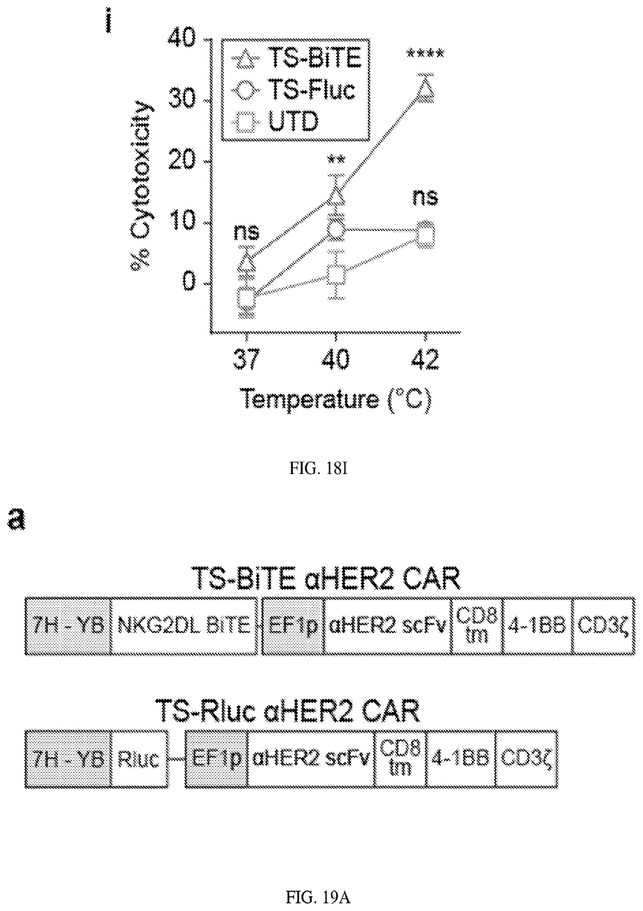

Heterogenous expression of antigens can lead to tumor escape from CAR T cells that are directed against a single antigen. It was therefore sought to determine whether heat-triggered expression of a bi-specific T cell engager (BiTE) targeting NKG2K ligands (NKG2DL)—which are upregulated on a wide range of cancers as well as suppressor cells⁷⁰⁻⁷³—could mitigate antigen escape. A previously described NKG2DL-BiTE containing CD3-recognition domains from the OKT3 antibody linked to the extracellular domain of the human NKG2D receptor was cloned. This vector (TS-BITE) included an Igκ leader sequence for BiTE secretion, a HisTag reporter, and a constitutive αCD19 CAR (FIG. 18a). After heat treatment, TS-BITE T cells were observed with positive staining by anti-HisTag antibodies compared to TS-Fluc control cells (FIG. 18b). TS-BITE T cells can undergo autocrine activation before BiTEs would engage bystander T cells for paracrine activation. To test this, a mixture of TS-BiTE Jurkat T cells were heated with untransduced cells as bystanders prior to co-incubation with NKG2DL+ CD19− K562 target cells (FIG. 18c-e) to isolate T cell activation by BiTE engagement without confounding factors due to CD19 CAR binding. Expression of the early activation marker CD69 on TS-BiTE Jurkat T cells was found to be significantly upregulated compared to bystander cells as heating durations were extended (red versus black) (FIG. 18f, g). By contrast, CD69 was minimally upregulated on bystander cells compared to untransduced (UTD) Jurkat T cells that were incubated with K562 cells and heated in separate wells as controls (black versus gray). These data provided support that TS-BITE T cells are primarily activated in an autocrine path. To quantify cytotoxicity from heat-triggered expression of BiTEs, primary human TS-BiTE αCD19 CAR T cells were co-incubated with NKG2DL+ CD19− K562 cells. In contrast to untransduced or TS-Fluc αCD19 CAR controls, TS-BITE αCD19 CAR T cells secreted increasing levels of Tₕ1 cytokines IFN-γ and TNF-α as temperatures were raised from 37 to 42° C. (FIG. 18h). Temperature-dependent increases in K562 cytotoxicity but not at 37° C. were observed compared to UTD controls, demonstrating lack of BiTE-induced killing at basal temperatures (FIG. 18i). These data showed that TS-BiTE αCD19 CAR T cells can be redirected to target antigen negative tumor cells that express of NKG2DL by thermal control.

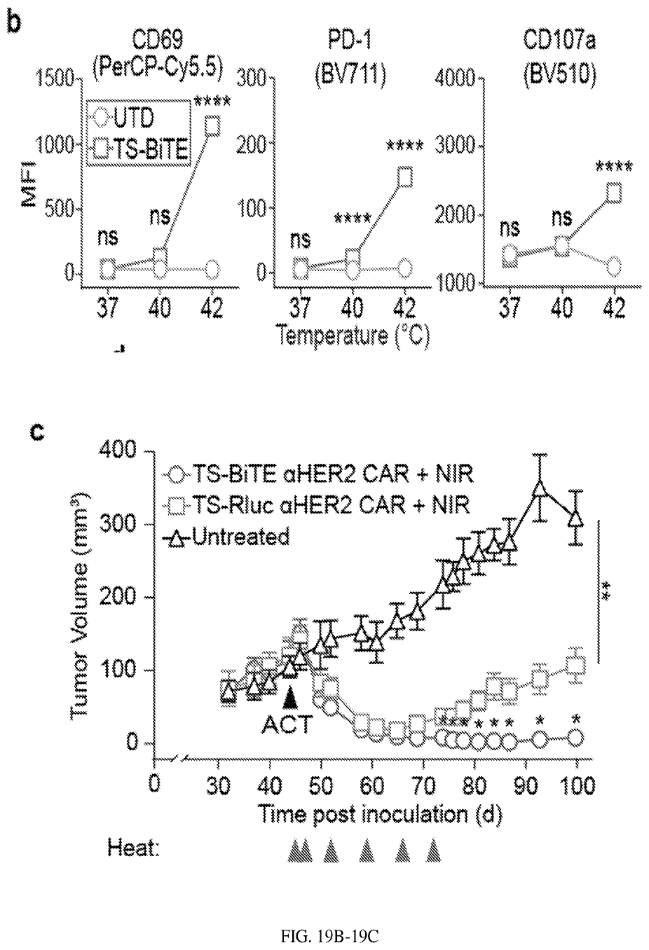

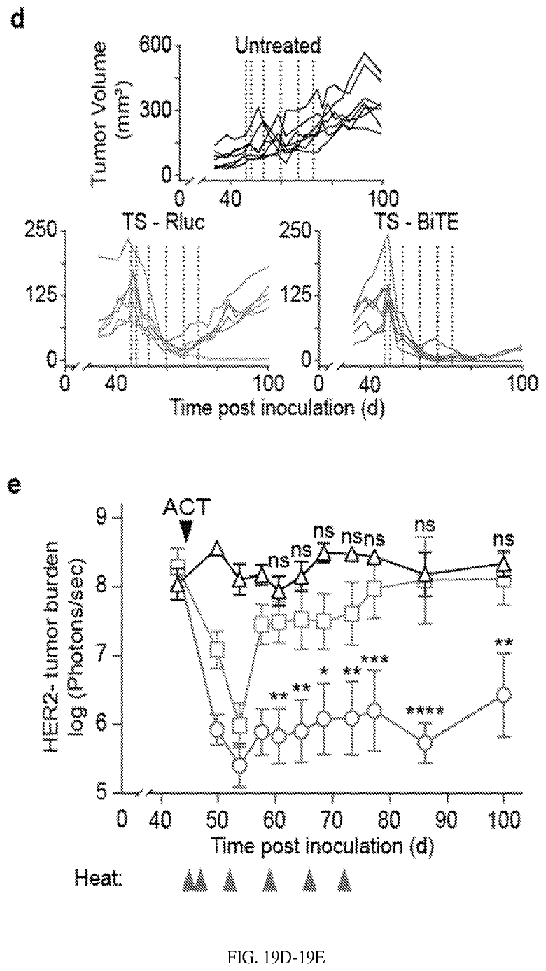

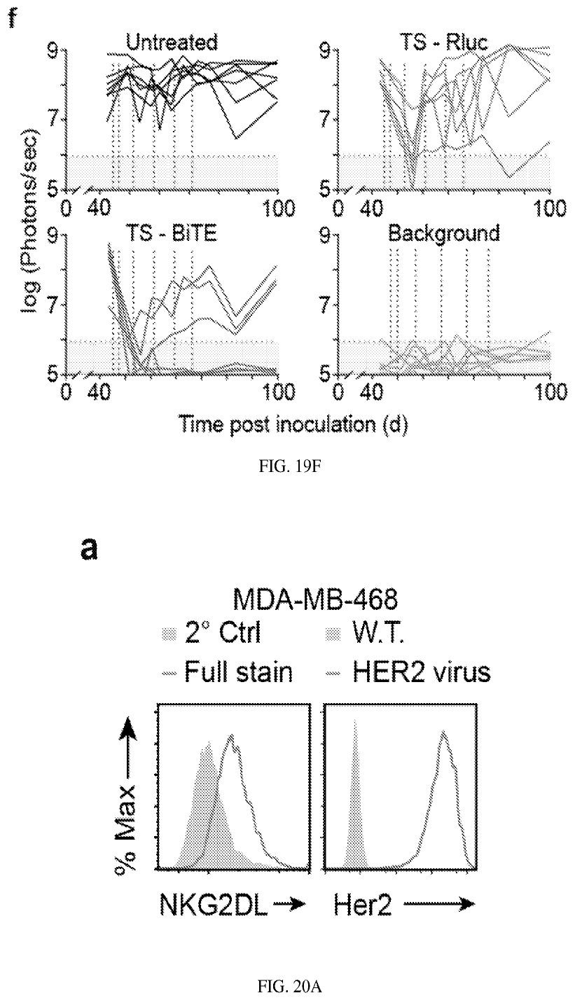

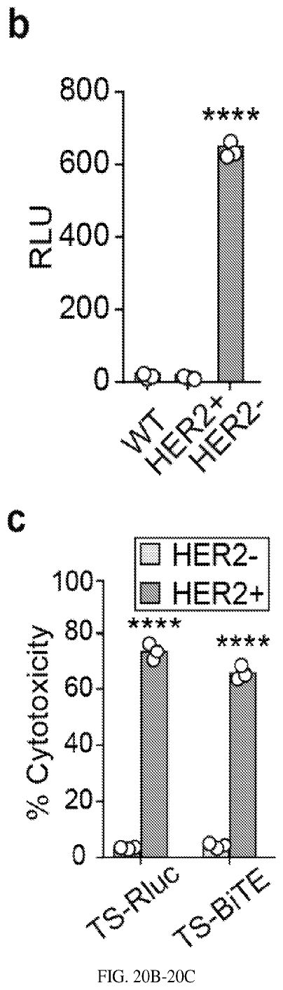

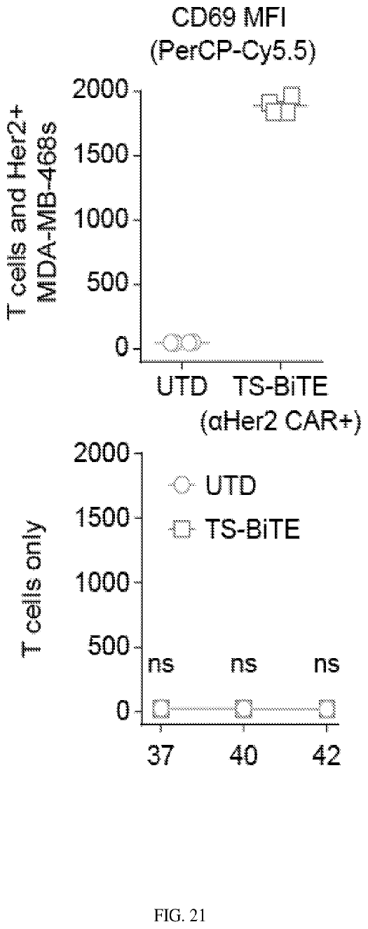

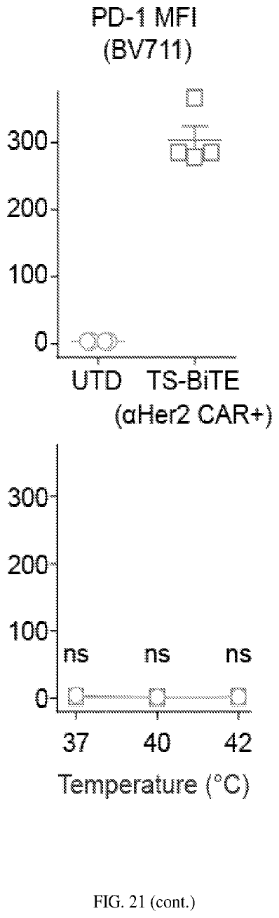

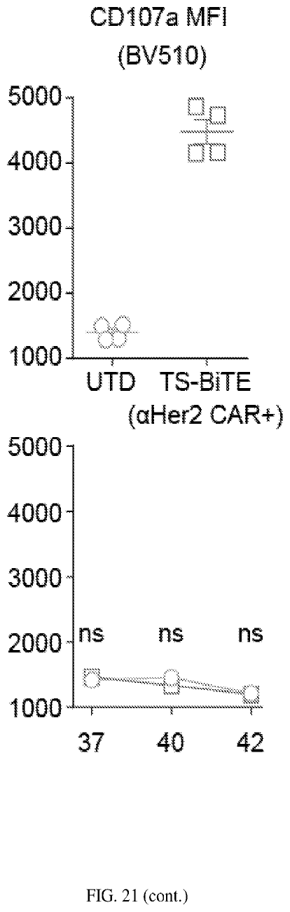

To test mitigation of antigen escape in vivo, a heterogenous model of breast cancer was developed comprising a mixture of HER2+ and HER2-MDA-MB-468 tumor cells. Endogenous expression of NKG2DL was verified in wild type cells and transduced them with either HER2 (FIG. 20a) or Fluc (FIG. 20b) to allow luminescent quantification of antigen negative cells in vivo. Both TS-BiTE or TS-Rluc αHER2 CAR T cells were confirmed to selectively target and kill HER2+ MDA-MB-468 cells (FIG. 19a; FIG. 20c). By contrast, targeting of HER2-cells required thermal treatment of TS-BiTE αHER2 CAR T cells as confirmed by temperature-dependent elevation of the activation markers CD69, PD-1, and CD107a (FIG. 19b; FIG. 21). To test whether thermal control of NKG2DL BiTE could treat tumors with heterogenous antigen expression, NSG mice were inoculated with HER2+ and HER2-MDA-MB-468 cells at a 3:1 ratio and transferred TS-BiTE or TS-Rluc αHER2 CAR T cells on day 44 when tumors were well-established and vascularized (˜110 mm³ average volume) (FIG. 19c). For approximately 40 days following ACT with longitudinal heating (days 45, 47, 52, 59, 66, and 72), significant tumor regression was observed in mice treated with either TS-BiTE or TS-Rluc αHER2 CAR T cells, the latter of which was attributed to killing of the HER2+ fraction of the tumors. However, by day 74, tumors from mice treated with TS-Rluc αHER2 CAR T cells began to relapse relative to TS-BiTE treated cohorts, resulting in tumors that were ˜12 times larger in volume on average by day 100. Tumors from 4 out of 6 TS-BiTE mice and 1 out of 6 TS-Rluc mice were undetectable by caliper measurements and palpation (FIG. 19d). To further corroborate these findings and determine whether relapse was attributable to outgrowth of antigen negative cells, tumor luminescence was quantified from HER2-cells and observed significant signal reduction in TS-BiTE compared to TS-Rluc groups by day 60 before tumor volumes began to diverge (FIG. 19e). residual disease in 1 mouse was observed from both the TS-BiTE and TS-Rluc groups that initially appeared to be a complete responder by caliper measurements but had luminescent signals that were above background, which was defined as two standard deviations above the average. 3 of 6 TS-BITE mice that had unpalpable tumors and luminescence within background levels for over ˜45 days was considered to be complete responders (FIG. 19f). These data demonstrate that thermal control of NKG2DL BiTE has the potential to mitigate antigen escape in tumors with heterogenous antigen expression.

### Discussion

The ability to better control engineered T cell activity within tumor sites has the potential to improve therapy against solid tumors. Here, a platform was developed for remote thermal control of T cell activity. To provide T cells the capacity to respond to heat, synthetic thermal gene switches were designed comprising arrays of heat shock elements upstream of a core promoter. This architecture eliminated sensitivity to non-thermal stresses such as hypoxia and its thermal response was tunable based on the number of HSEs or different core promoters. Importantly, negligible activation of the thermal gene switches was observed at temperatures ≤40° C. when T cells were incubated for over 24 hours, providing support that the temperature threshold for activation is higher than the range of typical fevers (˜38-40° C.) in patients with cytokine release syndrome (CRS), which would prevent T cell activation without a targeted thermal input.

The thermal control of T cell activity with an IL-15 superagonist and a NKG2DL BITE was demonstrated to enhance anti-tumor responses. Engineered T cells that constitutively express similar classes of molecules have demonstrated strong anti-tumor efficacy but their therapeutic applications are limited by off-tumor effects and toxicities in healthy tissues. Thus, targeted expression of these genes within tumors contains potent T cell activity and improve therapeutic outcomes. The thermal induction of transgenes was shown to be transient and reversible, and found that thermally activated T cells remained localized to the heated site when transgene expression was on, reducing the potential for off-target expression of transgenes. In K562 and syngeneic B16-F10 tumors, photothermal control of IL-15 SA expression by either αCD19 CAR or TCR-transgenic Pmel-1 T cells resulted in enhanced anti-tumor activity compared to adoptive transfer of T cells alone. It was further demonstrated that remote thermal control of a NKG2DL BiTE to mitigate antigen escape by allowing CAR T cells to target antigen negative tumor cells that express NKG2D ligands. In a mixed model of HER2+ and HER2-breast cancer, treatment with TS-BiTE αHER2 CAR T cells led to elimination of well-established tumors without detectable residual disease in 3 of 6 mice, or significantly delayed relapse compared to treatment with @HER2 CAR T cells that targeted a single antigen. In light of these results, a wide range of biologics are amenable for thermal control without potential loss of function due to protein misfolding or aggregation in T cells by heat stress.

Last, some of the conclusions from these studies are context specific. For example, in vitro experiments showing that BiTE activation occurs primarily by an autocrine mechanism can be affected by secretion rates, diffusion, and effector to bystander ratios as these parameters are tunable. Taken together, these results support remote thermal targeting of engineered T cell therapies to improve responses against solid tumors.

### Methods

### Plasmid Construction.

Synthetic thermal switches were produced as gene blocks by IDT and cloned into the Lego-C (Addgene plasmid #27348) or pMKO.1 (Imgenex, San Diego, CA) backbones. The core promoters were truncated immediately upstream of their previously described TATA boxes at their 5′-termini and at their translational start site on their 3′-termini. The genomic HSPA6 promoter was amplified from genomic DNA using PCR primers listed in a previous publication. The NKG2DL BiTE sequence (US20120294857A1) was described previously and modified to include an Igκ leader sequence to facilitate secretion from T cells as well as a HisTag for construct detection. This combined sequence was synthesized (ATUM) and cloned downstream of synthetic thermal gene switches. The IL-15 superagonist sequence was described previously and synthesized by ATUM without modification. The constitutive αCD19 CAR (U.S. Pat. No. 9,499,629B2) was kindly provided by Dr. Krishnendu Roy (Georgia Institute of Technology). The αHER2 CAR (US20180326032A1) was described previously⁹³. All unique materials can be made available by the corresponding author on reasonable request.

### Culture of Primary Human T Cells and Cell Lines.

CD19+ K562 (acquired from Dr. Yvonne Chen) and wild-type K562s (acquired from Dr. Krishnendu Roy) were cultured in Isocove's Modified Dulbecco's Medium (ThermoFisher #12440053) supplemented with 10% FBS (Fisher #16140071) and 10 U/ml Penicillin-Streptomycin (Life Technologies #15140-122). Raji cells were obtained from Dr. Krishnendu Roy and cultured in RPMI-1640 media supplemented with 10% FBS. MDA-MB-468 (ATCC, HTB-132) and B16-F10 (ATCC, CRL-6475) cells were cultured in Dulbecco's Modified Eagle Medium (Gibco #11995073) supplemented with 10% FBS (Fisher #16140071) and 10 U/ml Penicillin-Streptomycin (Life Technologies #15140-122). Primary Human CD3+ cells were obtained from anonymous donor blood after apheresis (AllCells) and were cryopreserved in 90% FBS and 10% DMSO until subsequent use. After thawing, cells were cultured in human T cell media comprised of X-VIVO 10 (Lonza #04-380Q), 5% human AB serum (Valley Biomedical #HP1022), 10 mM N-acetyl L-Cysteine (Sigma #A9165), and 55 μM 2-mercaptoethanol (Sigma #M3148-100ML) supplemented with 50 units/mL human IL-2 (Sigma #11147528001). Seven total donors were utilized for experimentation. FIG. 1 used donors 1, 2, 6 and 7; FIG. 5 used Donors 2 and 3; FIG. 9 used Donor 2; FIG. 13 used Donor 4; FIGS. 18 and 19 used Donor 2.

### Isolation and Expansion of Pmel-1 T Cells.

Splenocytes from Pmel-1 transgenic mice were depleted of red blood cells using RBC Lysis Buffer (Biolegend #420302) and cultured in complete medium with 100 units/mL recombinant human IL-2 (Sigma #11147528001) in the presence of 1 μg/mL hgp100₂₅₋₃₃ peptide for 2 days (Tufts University Core Facility). Splenocytes were resuspended at 10×10⁶ cells/mL in a media spiked with retrovirus at a multiplicity of infection of 10 and centrifuged for 90 minutes at 2000×g. Cells were cultured at a concentration of 1×10⁶ cells/mL in complete media supplemented with 100 units/mL IL-2 before intravenous transfer into C57BL/6J mice 6 days after isolation.

### Viral Production and Primary Human T Cell Transduction.

VSV-G pseudotyped lentivirus was produced via transfection of HEK 293T cells (ATCC, CRL-3216) using psPAX2 (Addgene #12260) and pMD2.G (Addgene #12259); viral supernatant was concentrated using PEG-it virus precipitation solution (System Biosciences LV825A-1) according to manufacturer instructions. Retrovirus was produced via transfection of HEK 293T cells (ATCC, CRL-3216) using pCL-Eco and p.MIKO.1 vector (Imgenex, San Diego, CA) encoding for the thermal switch circuit (TS-IL15); after 48 hours, viral supernatant was concentrated using Retro-Concentin retroviral concentration reagent (System Biosciences RV100A-1) according to manufacturer's instructions and frozen at −80° C. For viral transductions of primary human T cells, cells were thawed, incubated for 24 hours, and activated with Human T-Activator Dynabeads (Life Technologies #11131D) at a 3:1 bead:cell ratio for 24 hours. To transduce the activated T cells, concentrated lentivirus was added to non-TC treated 6-well plates which were coated with retronectin (Takara #T100B) according to manufacturer's instructions and spun at 1200×g for 90 min at room temperature. Following centrifugation, viral solution was aspirated and 2 mL of human T cells (250,000 cells/mL) in human T cell media containing 100 units/mL hIL-2 were added and spun at 1200×g for 60 min at 37° C. and moved to an incubator. Cells were incubated on a virus-coated plate for 24 hours prior to expansion and Dynabeads were removed 7 days after T cell activation. For cells flow-sorted prior to adoptive cell transfer, Dynabeads were added immediately after sorting at 3:1 ratios for 48 hours.

### Staining and Flow Cytometry.

To detect CAR expression, biotinylated CD19 (10 μg/mL; Acro Biosystems #CD9-H8259) and Streptavidin-APC (ThermoFisher #S868) were used according to manufacturer instructions. NKG2DL expression was assessed by staining with NKG2D-Fc chimera (10 μg/mL; Fisher 1299NK050) followed by an αFc secondary stain (Invitrogen #A-10631). NIR Live/Dead (ThermoFisher #L34976), CFSE (LifeTech #C34554) and CellTrace Violet (CTV; LifeTech #C34557) were used according to manufacturer instructions. Human Fc block (BD #564220) was used prior to staining with any antibodies. For intracellular staining for Granzyme B, intracellular fixation and permeabilization buffers (eBioscience #88-8823-88) were used according to manufacturer instructions with Brefeldin A being added ˜4 hours prior to staining. Antibodies for Granzyme B (GB12; ThermoFisher), CD69 (FN50; BD), CD4 (RPA-T4; BioLegend); hCD8 (RPA-T8; BioLegend), CD3 (UCHT1; BD), CD45 (HI30; BD), CD19 (HIB19; BioLegend), PD-1 (EH12.2H7, Biolegend), CD107a (H4A3, Biolegend), mCD8 (53-6.7, BioLegend), HER2 (24D2, Biolegend), and HisTags (4E3D10H2/E3; ThermoFisher) and human Fc (Invitrogen #A-10631) were all used at 1:100 dilutions.

In vitro luciferase and thermotolerance assays. Primary human T cells were heated in a thermal cycler and transferred to culture plates for incubation at 37° C. Unless otherwise noted, cellular supernatant was sampled for luciferase activity 24 hours after conclusion of thermal treatment. Non-thermal treatments were conducted by incubating engineered cells at indicated concentrations of CoCl₂ (Sigma #232696-5G) or CdCl₂ (Sigma #202908). When indicated, luminescence was compared to a ladder of recombinant Gaussia Luciferase (NanoLight #321-500) quantified using a Gaussia Luciferase Glow Assay Kit (ThermoFisher #16161) according to manufacturer's instructions. For viability and proliferation studies, primary human T cells were heated in the thermal cycler prior to assaying with an apoptosis detection kit (BD #556547) or CellTrace Violet (Fisher #C34571). Viability was assessed 24 hours after heating and gating strategies are depicted in FIG. 6. For migration studies, wild-type cells were added to the top insert of a transwell plate (Sigma #CLS3421) while CXCL12 (50 ng/mL, Peprotech #300-28A) was added to the lower chamber. Cells in lower chamber were counted by hemocytometer at indicated times.

### Cytotoxicity and T Cell Activation Assays.

For cytometric analysis, TS-CAR T cells were heated in a thermal cycler and co-incubated with K562 target cells at a 10:1 effector cell to target cell ratio for 24 hrs prior to staining as described above. For luciferase-based assays, K562s were luciferized with either Firefly luciferase (CD19+) or Renilla luciferase (CD19−) and incubated with effector cells after heating. Unless otherwise noted, a 10:1 effector to target ratio was used. After incubation, either D-luciferin (Fisher #LUCK-2G; 150 μg/mL read concentration) or Rluc substrate (VWR #PAP1232; 17 UM read concentration) was added to the sample. Maximum cytotoxicity was defined as luminescent signal from wells containing only media while no cytotoxicity was defined by wells containing only target cells. Supernatant was collected after incubation and assayed for cytokines using the human Th1/Th2/Th17 CBA kit (BD #560484). IL-15 superagonist was quantified using the human IL-15/IL-15R alpha complex DuoSet ELISA (R&D Systems DY6924). For BiTE experiments with primary human T cells, two heat treatments (42° C., 30 minutes) separated by 6 hours were applied to T cells prior to incubation with target cells. The ability of engineered T cells to kill tumor target cells was also measured by lactate dehydrogenase (LDH) release assay. Briefly, engineered T cells were co-cultured with target cells at a 2:1 effector cell to target ratio for 24 h in a 96-well plate. Then LDH release was measured by the LDH-cytotoxicity Assay Kit (Fluorometric) (Abcam #197004) according to the manufacturer's instructions.

### IL-15 Superagonist Dynabead Experiment:

Wild-type primary human T cells were labeled with CFSE and incubated with either heated or unheated TS-IL15 cells. Beads were added at a 10:1 T cell to bead ratio that was determined not to induce strong proliferation in untransduced T cells without cytokine support (FIG. 15). CFSE labeling allowed discrimination from TS-IL15 cells (FIG. 16) and proliferation and division indices were calculated in FlowJo using the Proliferation tool.

### Animals:

NSG mice were bred and housed in the Georgia Tech Physiological Research Laboratory (GT PRL) prior to use at an age of 8 to 16 weeks. C57BL/6 mice and transgenic Pmel-1 mice (B6.Cg-Thy1a/Cy Tg(TcraTcrb)8Rest/J) were purchased from Jackson Laboratories. 6 to 8 week old C57BL/6 and Pmel-1 mice were used at the outset of experiments. All animal protocols were approved by Georgia Tech IACUC (protocols no. A100190 and A100191). All authors have complied with relevant ethical regulations while conducting this study.

### Photothermal Heating and In Vivo Bioluminescence Imaging:

AuNRs were purchased from Nanopartz (#A12-10-808-CTAB-500) and pegylated (Laysam Bio ##MPEG-SH-5000-5g) to replace the CTAB coating. These AuNRs were intravenously injected into tumor-bearing mice (10 mg/kg) ˜24-48 hrs before adoptive transfer of T cells. Mice were anesthetized with isoflurane gas, and target sites were irradiated using an 808 nm laser (Coherent) under guidance of a thermal camera (FLIR model 450 sc). Fluc activity was measured using an IVIS Spectrum CT (Perkin Elmer) ˜5 minutes after intravenous injections or 20 minutes after intraperitoneal injection of D-luciferin (Fisher #LUCK-2G). The detection limit was identified by calculating the mean±2 standard deviations of background measurements.

### Adoptive Cell Transfer (ACT) Experiments:

NSG mice were inoculated subcutaneously with 5×10⁶ Raji or K562 cell lines after the site was shaved and sterilized using an isopropyl wipe (GT PRL) for 9 days before ACT. For heterogenous expression of HER2, 5×10⁵ MDA-MB-468 cells with a ratio of 1:3 HER2− to HER2+ were inoculated for 44 days before ACT. Engineered primary human T cells were injected via tail vein in 200 μL sterile saline. For B16 tumor models, 5×10⁵ B16F10 melanoma cells were inoculated in the flanks of C57BL/6 mice. Mice were sub-lethally lymphodepleted by total body irradiation (100 cGy/minute for 5 minutes) 8 days after tumor cell inoculation. Engineered Pmel-1 T cells (6×10⁶ cells) were administered by i.v. injection at day 9. Mice were intraperitoneally dosed with 2×10⁵ units recombinant human IL-2 (Peprotech #200-02) twice daily at least 10 hours apart for total 6 dose. All mice received pegylated AuNRs intravenously via tail vein ˜24 hours prior to adoptive transfer of human T cells. 25 hours after ACT, photothermal heat treatments were administered and monitored as described above.

### Software and Statistical Analysis.

All results are presented as mean, and error bars depict SEM. Statistical analysis was performed using GraphPad Prism statistical software. For all graphs, * p<0.05, **p<0.01. ***p<0.001, ****p<0.0001, ns=not significant. Flow cytometry data were analyzed using FlowJo X (FlowJo, LLC). In vitro luminescent data were collected with Gen5 2.07 (Biotek). In vivo luminescence data were collected and analyzed with Living Image 4.4.5 (PerkinElmer). Flow-cytometry data were collected with BD FACSDIVA v8 (BD Biosciences). Thermal imaging data were acquired and analyzed using Research IR Max (FLIR). Figures were designed in Adobe Illustrator.

### Example 2

Immune therapies have immense therapeutic capabilities and are used to treat a myriad of ailments such as asthma, graft rejection, and cancer. Of note, chimeric antigen receptor (CAR) T cell therapies have resulted in durable longterm survival in certain types of cancer patients with B cell malignancies. However, their effectiveness in treating solid tumors have been limited by factors such as tumor heterogeneity and severe immunosuppression. Potent immunomodulators such as cytokines (IL-2, -12, -15), growth factors (Flt3L, IGF-IR), or chemokines (CXCL12, CCL 19) can potentiate immune cell therapies to transiently regulate immune function. However, conventional delivery mechanisms reliant on systemic administration are associated with poor pharmacokinetics and numerous immune related adverse effects (irAEs) including on-target, off-tumor toxicity, cytokine-release syndrome, neurotoxicity, and in some instances, life-threatening autoimmunity. As immune therapies are developed for more disease indications, creating strategies to safely deliver immunomodulators with spatial precision will be critical to enhance systemic responses while mitigating irAEs. Here, we introduce a noninvasive method to spatially control T cell mediated drug delivery at distinct anatomical sites including but not limited to the brain, spleen, lymph nodes, tumor, kidney, stomach, intraperitoneal space, lungs, heart, liver, pancreas, and bladder. In contrast to drug delivery by passive diffusion, immune cells can infiltrate deep within tissue, target diseased sites, and home to lymphoid organs, thus providing opportunities to engineer immune cells both as therapy and as delivery vehicles to improve therapeutic efficacy and mitigate irAEs. We describe a platform wherein cells are engineered to controllably deliver therapeutic molecules including, but not limited to CARs, cytokines, chemokines, transcription factors, and nucleases under conditional control by thermal cues that can be spatially deposited by various mechanisms (e.g., focused ultrasound, light, radiation, etc.). We illustrate spatial control of engineered cells and demonstrate enhanced therapeutic efficacy attributed to local delivery of immunomodulatory molecules. Using numerous preclinical models, we show 1) spatial delivery of reporter molecules using engineered T cells as drug delivery vehicles to various anatomical sites, 2) enhanced antitumoral therapy attributed to local delivery of cytokines, and 3) mitigation of tumor outgrowth resultant of tumor heterogeneity. Taken together, this platform is a modular approach to locally deliver immunomodulatory molecules and enhance cell-based therapies to overcome challenges associated with systemic treatments while mitigating adverse, off-target events.

### Spatial Control of Engineered Immune Cells

In one implementation, we engineered a synthetic thermal gene switch (TS) comprised of a DNA nucleotide sequence encoding a series of heat shock elements (derived from various species including but not limited to H. sapiens, M. musculus, C. dromedarius, or D. rerio) upstream of a naturally or synthetically derived core promoters for transgene activation upon mild hyperthermia (40-44° C.). In one embodiment, we combined the TS with focused ultrasound (FUS) as a trigger to deliver proteins upon mild hyperthermia in various anatomical organs. T cells were virally transduced with a transgene encoding a thermal switch driving Firefly luciferase or Gaussia luciferase (TS-Fluc, SEQ ID 22 or TS-Gluc, SEQ ID 26) and adoptively transferred into mice. FUS was used to locally deposit heat and deliver molecules in the brain, tumor, or lymph node. See attached manuscript for additional data, which includes local delivery mediated by near infrared (NIR) light.

### Local Delivery of Cytokines by Engineered Immune Cells Enhances Immune Therapies.

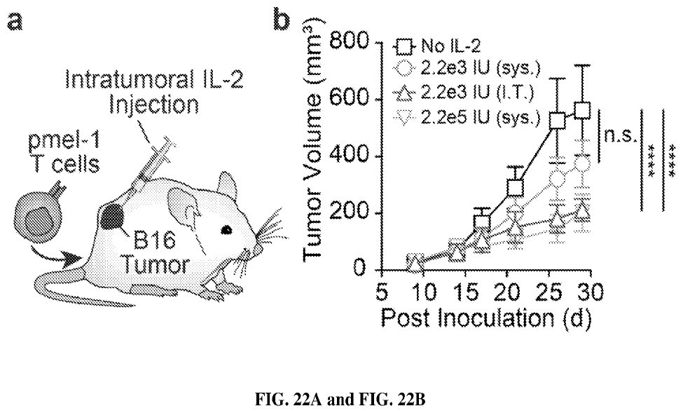

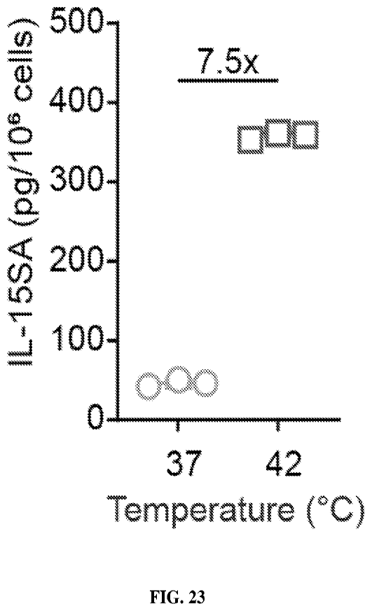

Spatial control of immunomodulatory genes such as those that encode for stimulatory (e.g., IL-2, -12, -15, TNFα, IFNγ, etc.), inhibitory (e.g., IL-6, -10, TGFβ, etc.), and chemotactic (e.g., CXCL12, CCL2, CCL19, etc.) molecules can enhance therapeutic outcomes over systemically administered molecules (FIG. 22). In one embodiment, dendritic cells were transduced with a transgene encoding thermally driven production of IL-15SA (TS-IL15SA SEQ ID 23) and heated using a thermocycler with a pulsed profile for 30 minutes (66% duty cycle). 12 hours post heat, IL-15SA production by the dendritic cells was quantified via an ELISA (FIG. 23).

### Local Delivery of Recombinant Proteins Augment Antitumoral Activity

Spatial control can also be implemented to modulate production of recombinant proteins including but not limited to antibodies and nanobodies (e.g. αPDL1, αCTLA-4, «IL-6r), transcription factors (e.g., NFAT, NFκB, T-bet), caspases (e.g. caspase 3, caspase 8), or bispecific T cell engagers (BiTEs) (e.g. NKG2DL, EGFRVIII, CD19 BiTEs).

1. A promoter construct comprising the following regions:
a) one or more heat shock elements;
b) a core promoter; and
c) a gene of interest.

2. The promoter construct of claim 1, wherein said promoter requires thermal activation between 40° C.-45° C.

3-8. (canceled)

9. The promoter construct of claim 1, wherein promoter construct is activated by a light source.

10. (canceled)

11. The promoter construct of claim 9, wherein the light source is a near infrared laser.

12. The promoter construct of claim 1, wherein the heat shock element is repeated 2, 3, 4, 5, 6, 7, or more times.

13. The promoter construct of claim 1, wherein the heat shock element comprises nGAAnnTTCnnGAAn.

14. The promoter construct of claim 13, wherein the one or more heat shock elements comprises the nucleotide sequence of any one of SEQ ID NOS:2-9.

15. The promoter construct of claim 1, wherein the core promoter comprises a heat shock protein transcription start site.

16. The promoter construct of claim 15, wherein the core promoter comprises the heat shock protein transcription start site of HSPA1A, HSPH1, HSPB1, HSPA6, or YB.

17. The promoter construct of claim 15, wherein the core promoter comprises any one of the following nucleotide sequences SEQ ID NOS: 10-13.

18. The promoter construct of claim 1, wherein the one or more heat shock elements and core promoter together comprises the nucleotide sequence of any one of SEQ ID NOS: 14-21.

19. The promoter construct of claim 1, wherein the gene of interest encodes:
a. a reporter protein;
b. an immunomodulating agent;
c. a bispecific T cell engager antibody;
d. a chimeric antigen receptor;
e. a recombinant T cell receptor,
or any combination thereof.

20-21. (canceled)

22. The promoter construct of claim 19, wherein the immunomodulating agent is 1) a cytokine selected from the group consisting of IL-1β, IL-2, IL-4, IL-6, IL-8, IL-10, IL-12, IL-15, IL-18, IL-21, IL-22, IFN-γ, TNF-α, TGF-β, and/or LIF; or 2) a chemokine selected from the group consisting of CCL2, CCL1, CCL19, CCL22, CXCL12, CCL17, MIP-1α, MCP-1, GRO/KC, CSCL12, and/or CXCR3.

23-24. (canceled)

25. The promoter construct of claim 19, wherein the bispecific T cell engager antibody comprises an anti-CD-3 binding domain and an NKG2D receptor extracellular domain.

26. The promoter construct of claim 1, comprising the nucleotide sequence of any one of SEQ ID NOS: 1, 23, and 24.

27. A vector comprising the promoter construct of claim 1.

28. (canceled)

29. An immune cell comprising the promoter construct of claim 1.

30. The immune cell of claim 29, wherein the immune cell is a T cell, a NK cell, recombinant TCR T cell, CAR T cell, or CAR NK cell.

31-32. (canceled)

33. A kit comprising the promoter construct of claim 1, and further comprising a heating element to activate the promoter construct.

34. A method of treating a cancer in a subject comprising administering to the subject the promoter construct of claim 1.

35. A method of treating a cancer in a subject comprising i) administering to the subject a thermally controlled immune cell comprising a promoter construct; wherein said promoter construct comprises one or more heat shock elements; a core promoter; and a gene of interest and ii) activating the thermally controlled cell with a heating element.

36. The method of treating a cancer of claim 35, wherein the heat shock element of the thermally controlled immune cell comprises nGAAnnTTCnnGAAn.

37. The method of claim 35, wherein the thermally controlled immune cell is a T cell or NK cell.

38. The method of treating a cancer of claim 35, wherein the gene of interest comprises a chimeric antigen receptor, an immunomodulating agent; a bispecific T cell engager (BiTE), a recombinant T cell receptor, or any combination thereof.

39. The method of treating a cancer of claim 35, wherein the thermally controlled immune cell activates at temperatures ranging from 40° C.-45° C.

41-46. (canceled)

47. The method treating a cancer of claim 35, further comprising administering to the subject an anti-PD1 immunotherapy or anti-PD-L1 immunotherapy.

48-50. (canceled)
